(19) United States
(12) Patent Application Publication
(10) Pub. No.: US 2025/0376427 A1
(43) Pub. Date: Dec. 11, 2025

(54) COMPOSITIONS AND METHODS FOR THE PURIFICATION OF SQUALENE

(71) Applicant: Amyris, Inc., Emeryville, CA (US)

(72) Inventors: Seham AFAGHANI, Emeryville, CA (US); Binita BHATTACHARJEE, Emeryville, CA (US); Greg Oniel BUCHANAN, Emeryville, CA (US); Steven Charles DEFINA, Emeryville, CA (US); Joshua R. GURR, Emeryville, CA (US); Ronald HENRY, Emeryville, CA (US); Stanley HERRMANN, Emeryville, CA (US); Paul W. HILL, Emeryville, CA (US); Elisa M. RODRIGUEZ PORCEL, Emeryville, CA (US); Daniel SEYEDEBRAHIMI, Emeryville, CA (US); Rudy SITHIRATH, Emeryville, CA (US)

(21) Appl. No.: 18/876,576

(22) PCT Filed: Jun. 23, 2023
(86) PCT No.: PCT/US2023/026063

§ 371 Date: Dec. 18, 2024

Related U.S. Application Data
(60) Provisional application No. 63/355,303, filed on Jun. 24, 2022.

(51) Int. Cl. C07C 11/21; A61K 31/01; A61K 39/00; A61K 39/12; C07C 7/04; C07C 7/10; C07C 7/12; C12N 1/16; C12R 1/645

(52) U.S. Cl. CPC C07C 11/21; A61K 31/01; A61K 39/12; C07C 7/04; C07C 7/10; C07C 7/12; C12N 1/16; A61K 2039/55511; C12R 2001/645

(57) ABSTRACT

The present disclosure provides compositions and methods for producing high-purity squalene from a squalene source, for example from host cells, such as yeast cells that are capable of synthesizing squalene. Further provided herein are compositions containing high-purity squalene, as well as methods of using the same in pharmaceutical formulations, such as in adjuvant formulations for use in vaccines.

### BACKGROUND OF THE INVENTION

Squalene, farnesene, and farnesene derivatives, such as farnesol and farnesyl acetate, are commercially significant isoprenoid compounds that have found use in a variety of applications. Squalene is a naturally occurring, 30-carbon organic compound produced by animals and plants that, to date, has primarily been obtained from shark liver oil. Squalene has many utilities. Since squalene is commonly generated by human sebaceous glands, squalene is often used in cosmetic and personal care products for topical skin lubrication and protection. Squalene is also an important ingredient in immunological adjuvants that are administered in conjunction with a vaccine. Adjuvants that contain squalene have shown the ability to augment a patient's immune response, enhancing the effectiveness of the corresponding vaccine. In some instances, because of this increased response, the amount of antigen included in a vaccine can be reduced substantially, while still maintaining immunoprotection.

Despite the benefits associated with squalene, there remains a need for improved methods of producing squalene, particularly in a renewable manner and with high purity. In light of the use of squalene in pharmaceutical compositions that are intended for administration to human subjects, there is a longstanding need for improved methods of generating squalene in a form that is substantially free of impurities.

### SUMMARY OF THE INVENTION

The present disclosure provides compositions and methods for producing squalene from renewable resources, such as from host cells (e.g., yeast cells) capable of synthesizing squalene upon fermentation, as well as compositions and methods for isolating squalene from such cells. The compositions and methods described herein address a problem that has been particularly challenging to the field of synthetic biology: how to sustainably obtain squalene from fermented host cells while recovering the squalene in a form that is substantially free of concomitant cellular impurities. Although squalene can be produced from host cells (e.g., yeast cells) that express the enzymes involved in squalene biosynthesis, the purification of squalene from such cells has been a significant challenge, particularly given that squalene is sequestered intracellularly and is not secreted to an appreciable extent. Accordingly, in order to access the squalene produced from fermented host cells, the host cells are generally homogenized, a process that involves lysing the cells to release intracellular components into extracellular media. Given the complex mixture of materials that are released during this process, purifying squalene from this matrix has posed significant difficulties.

The present disclosure is based, in part, on the surprising discovery that a unique combination of extraction, evaporation, and chromatography steps described herein results in squalene compositions having a level of purity not previously achieved using synthetic biology. The sections that follow provide a description of the compositions and methods that can be used to obtain squalene from a squalene source, such as squalene-producing host cells (e.g., yeast cells) with elevated purity.

In an aspect, the disclosure provides a method of isolating squalene from a squalene source including extracting the squalene from the squalene source; optionally evaporating the squalene resulting from (a); and purifying the squalene resulting from (b) by way of chromatography.

In an aspect, the disclosure provides a method of making squalene including (a) providing a squalene source; (b) extracting the squalene from a squalene source; (c) optionally evaporating the squalene resulting from (b); and (d) purifying the squalene resulting from (c) by way of chromatography.

In some embodiments, the squalene source is a fermentation source. In some embodiments, the fermentation source comprises yeast. In some embodiments, the squalene source is a plant source. In some embodiments, the plant source comprises olive, soybean, grape seed, grape, hazelnut, peanut, corn, amaranth, rice, wheat germ, coriander, sesame, or sunflower. In some embodiments, the squalene source is an animal source. In some embodiments, the squalene source is a fungi source. In some embodiments, the fermentation source comprises a stramenopile source. In some embodiments, the stramenopile source comprises algae.

In an aspect, the disclosure provides a method of isolating squalene from a fermentation composition. The fermentation composition may be one that has been produced, for example, by culturing a population of host cells capable of producing squalene in a culture medium and under conditions suitable for the host cells to produce squalene. In some embodiments, the method includes extracting the squalene from the fermentation composition. In some embodiments, the extracting comprises one or more of: homogenization, centrifugation, solvent extraction, and demulsification. The extraction step may include one or more of: (i) homogenizing the fermentation composition, (ii) separating the homogenized fermentation composition resulting from (i) into sediment and supernatant by way of centrifugation, (iii) demulsifying supernatant obtained from (ii), and (iv) separating the demulsified supernatant resulting from (iii) into an aqueous component and an oil component. The method may further include evaporating the squalene resulting from the extraction step. In some embodiments, the method further includes purifying the squalene resulting from the evaporation step by way of chromatography, such as an alumina or a silica chromatography process described herein. In some embodiments, the extraction step includes one or more, or all, of: (i) homogenizing the fermentation composition (e.g., by way of a homogenization technique described herein), (ii) solvent extraction of the fermentation composition, (iii) centrifugation of the fermentation composition, followed, e.g., by (a) solvent extraction of the ensuing pellet and/or (b) solvent extraction of the ensuing supernatant, which may, optionally, be demulsified (e.g., by way of a demulsification technique described herein).

In another aspect, the disclosure provides a method of making squalene. The method may include, for example, culturing a population of host cells capable of producing squalene in a culture medium and under conditions suitable for the host cells to produce squalene, thereby producing a fermentation composition. The method may further include extracting the squalene from the fermentation composition. In some embodiments, the extracting includes one or more of: homogenization, centrifugation, solvent extraction, and demulsification. In some embodiments, the extraction step includes one or more of: (i) homogenizing the fermentation composition, (ii) separating the homogenized fermentation composition resulting from (i) into sediment and supernatant by way of centrifugation, (iii) demulsifying supernatant obtained from (ii), and (iv) separating the demulsified supernatant resulting from (iii) into an aqueous component and an oil component. In some embodiments, the method further includes evaporating the squalene resulting from the extraction step. In some embodiments, the method further includes purifying the squalene resulting from the evaporation step by way of chromatography, such as an alumina or a silica chromatography process described herein.

In some embodiments, the extracting includes homogenizing the fermentation composition. In some embodiments, prior to homogenizing the fermentation composition, the fermentation composition is diluted in water to a final concentration of from about 20% to about 40% solid material (v/v) (e.g., from about 20% to about 35% solid material (v/v), from about 20% to about 30% solid material (v/v), from about 20% to about 25% solid material (v/v), from about 25% to about 40% solid material (v/v), from about 30% to about 40% solid material (v/v), or from about 35% to about 40% solid material (v/v)). In some embodiments, prior to homogenizing the fermentation composition, the fermentation composition is diluted in water to a final concentration of from about 30% to about 35% solid material (v/v) (e.g., about 30%, 31%, 32%, 33%, 34%, or 35% solid material (v/v)).

In some embodiments, the fermentation composition is homogenized in one or more steps. In some embodiments, the fermentation composition is homogenized in from one to five steps (e.g., in one step, two steps, three steps, four steps, or five steps). In some embodiments, the fermentation composition is homogenized in from one to three steps (e.g., in one step, two steps, or three steps). In some embodiments, the fermentation composition is homogenized in two steps.

In some embodiments, each homogenization step includes homogenizing the fermentation composition at a pressure of from about 400 bar to about 1,200 bar (e.g., at a pressure of from about 500 bar to about 1,200 bar, from about 600 bar to about 1,200 bar, from about 700 bar to about 1,200 bar, from about 800 bar to about 1,200 bar, from about 900 bar to about 1,200 bar, or from about 1,000 to about 1,200 bar). In some embodiments, each homogenization step includes homogenizing the fermentation composition at a pressure of from about 800 bar to about 1,000 bar (e.g., at a pressure of from about 800 bar to about 950 bar, from about 800 bar to about 900 bar, from about 800 bar to about 850 bar, from about 850 bar to about 1,000 bar, or from about 900 bar to about 1,000 bar). In some embodiments, each homogenization step may include homogenizing the fermentation composition at a pressure of about 900 bar.

In some embodiments, each homogenization step includes homogenizing the fermentation composition at a temperature from about 5° C. to about 70° C. (e.g., about 5° C., 10° C., 15° C., 20° C., 25° C., 30° C., 35° C., 40° C., 45° C., 50° C., 55° C., 60° C., 65° C., or 70° C.). In some embodiments, each homogenization step includes homogenizing the fermentation composition at ambient temperature. In some embodiments, each homogenization step includes homogenizing the fermentation composition at pH of from about 3 to about 9 (e.g., about 3 to about 8, about 3 to about 7, about 3 to about 6, about 3 to about 5, about 3 to about 4, about 4 to about 9, about 5 to about 9, about 6 to about 9, about 7 to about 9, about 8 to about 9, or about 5 to about 8). In some embodiments, each homogenization step includes homogenizing the fermentation composition at native pH.

In some embodiments, the extracting includes separating the homogenized fermentation composition resulting from (i) into sediment and supernatant, for example, by way of solid-liquid centrifugation. In some embodiments, prior to the centrifugation of the fermentation composition resulting from (i), the fermentation composition is heated to a temperature of from about 18° C. to about 75° C. (e.g., at a temperature of about 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or 75° C.).

In some embodiments, prior to centrifugation of the fermentation composition resulting from (i), the fermentation composition resulting from (i) is diluted in water to a final concentration of from about 20% to about 30% solid material (v/v) (e.g., about 20%, 21%, 22%, 23%, 24%, 25%, 26%, 27%, 28%, 29%, or 30% solid material (v/v)). In some embodiments, the fermentation composition resulting from (i) is diluted in water to a final concentration of about 25% solid material (v/v) prior to the centrifugation. In some embodiments, the fermentation composition resulting from (i) is centrifuged. In some embodiments, the fermentation composition resulting from (i) is centrifuged with a continuous centrifuge. In some embodiments, the fermentation composition resulting from (i) is centrifuged at a rate of from about 3,000 revolutions per minute (rpm) to about 5,000 rpm (e.g., from about 3,500 rpm to about 5,000 rpm, from about 4,000 rpm to about 5,000 rpm, from about 4,500 rpm to about 5,000 rpm, from about 3,000 rpm to about 4,500 rpm, from about 3,000 rpm to about 4,000 rpm, or from about 3,000 rpm to about 3,500 rpm). In some embodiments, the fermentation composition resulting from (i) is centrifuged at a rate of about 4,100 rpm. In some embodiments, the fermentation composition resulting from (i) is centrifuged at a rate of from about 10×G to about 10,000×G (e.g., from about 10×G to about 8,000×G, from about 10×G to about 6,000×G, from about 10×G to about 4,000×G, from about 10×G to about 2,000×G, from about 10×G to about 100×G, 100×G to about 10,000×G, from about 1,000×G to about 10,000×G, from about 3,000×G to about 10,000×G, from about 5,000×G to about 10,000×G, from about 7,000×G to about 10,000×G, or 9,000×G to about 10,000×G).

In some embodiments, the fermentation composition resulting from (i) is centrifuged for from about 5 minutes to about 30 minutes (e.g., from about 5 minutes to about 25 minutes, from about 5 minutes to about 20 minutes, from about 5 minutes to about 15 minutes, from about 5 minutes to about 10 minutes, from about 10 minutes to about 30 minutes, from about 15 minutes to about 30 minutes, from about 20 minutes to about 30 minutes, or from about 25 minutes to about 30 minutes). In some embodiments, the fermentation composition resulting from (i) is centrifuged for about 15 minutes.

In some embodiments, the extracting includes demulsifying the supernatant obtained from (ii). In some embodiments, the demulsifying includes contacting the supernatant obtained from (ii) with a surfactant. In some embodiments, the surfactant is selected from DOWFAX® 2A1, DOWFAX® 3B2, DOWFAX® 8390, DOWFAX® C6L, DOWFAX® C10L, TRITON® QS-15, TRITON® XN-45S, and TERGITOL® L62, or any combination thereof. In some embodiments, the surfactant is DOWFAX® 2A1.

In some embodiments, the surfactant is added to the supernatant obtained from (ii) to a final concentration of from about 0.01% to about 5% (v/v) (e.g., from about 0.01% to about 4% (v/v), from about 0.01% to about 3% (v/v), from about 0.01% to about 2% (v/v), from about 0.01% to about 1% (v/v), from about 0.01% to about 0.1% (v/v), from about 0.1% to about 5% (v/v), from about 1% to about 4% (v/v), from about 1% to about 3% (v/v), or from about 1% to about 2% (v/v)). In some embodiments, the surfactant is added to the supernatant obtained from (ii) to a final concentration of from about 1% to about 2% (v/v) (e.g., about 1.1% (v/v), 1.2% (v/v), 1.3% (v/v), 1.4% (v/v), 1.5% (v/V), 1.6% (v/v), 1.7% (v/v), 1.8% (v/v), 1.9% (v/v), or 2% (v/v)).

In some embodiments, the demulsifying is performed at a pH of from about 6 to about 8 (e.g., a pH of from about 6 to about 7.5, from about 6 to about 7, from about 6 to about 6.5, from about 6.5 to about 8, from about 7 to about 8, or from about 7.5 to about 8). In some embodiments, the demulsifying is performed at a temperature of from about 50° C. to about 90° C. (e.g., from about 50° C. to about 80° C., from about 50° C. to about 70° C., from about 50° C. to about 60° C., from about 60° C. to about 90° C., from about 70° C. to about 90° C., or from about 80° C. to about 90° C.). In some embodiments, the demulsifying is performed at a temperature of about 70° C.

In some embodiments, the extracting includes separating the demulsified supernatant resulting from (iii) into an aqueous component and an oil component. In some embodiments, the demulsified supernatant resulting from (iii) is separated into an aqueous component and an oil component by way of liquid-liquid centrifugation. In some embodiments, the liquid-liquid centrifugation is performed in one or more steps (e.g., in from one to five steps, such as in one, two, three, four, or five steps). In some embodiments, the liquid-liquid centrifugation is performed in two steps. In some embodiments, in a first liquid-liquid centrifugation step, the demulsified supernatant resulting from (iii) is centrifuged at a temperature of from about 50° C. to about 90° C. (e.g., from about 50° C. to about 80° C., from about 50° C. to about 70° C., from about 50° C. to about 60° C., from about 60° C. to about 90° C., from about 70° C. to about 90° C., or from about 80° C. to about 90° C.). In some embodiments, in the first liquid-liquid centrifugation step, the demulsified supernatant resulting from (iii) is centrifuged at a temperature of about 70° C.

In some embodiments, in the first liquid-liquid centrifugation step, the demulsified supernatant resulting from (iii) is centrifuged at a rate of from about 3,000 rpm to about 5,000 rpm (e.g., about 3,500 rpm to about 5,000 rpm, about 4,000 rpm to about 5,000 rpm, about 4,500 rpm to about 5,000 rpm, about 3,000 rpm to about 4,500 rpm, about 3,000 rpm to about 4,000 rpm, or about 3,000 rpm to about 3,500 rpm). In some embodiments, in the first liquid-liquid centrifugation step, the demulsified supernatant resulting from (iii) is centrifuged at a rate of about 4,100 rpm. In some embodiments, in the first liquid-liquid centrifugation step, the demulsified supernatant resulting from (iii) is centrifuged with a continuous centrifuge.

In some embodiments, in the first liquid-liquid centrifugation step, the demulsified supernatant resulting from (iii) is centrifuged for from about 5 minutes to about 30 minutes (e.g., about 5 minutes to about 25 minutes, about 5 minutes to about 20 minutes, about 5 minutes to about 15 minutes, about 5 minutes to about 10 minutes, about 10 minutes to about 30 minutes, about 15 minutes to about 30 minutes, about 20 minutes to about 30 minutes, or about 25 minutes to about 30 minutes). In some embodiments, in the first liquid-liquid centrifugation step, the demulsified supernatant resulting from (iii) is centrifuged for about 15 minutes.

In some embodiments, in a second liquid-liquid centrifugation step, the demulsified supernatant resulting from (iii) is centrifuged at a temperature of from about 25° C. to about 70° C. (e.g., about 25° C. to about 60° C., about 25° C. to about 50° C., about 25° C. to about 40° C., about 25° C. to about 30° C., about 30° C. to about 70° C., about 40° C. to about 70° C., or about 50° C. to about 70° C.)). In some embodiments, in the second liquid-liquid centrifugation step, the demulsified supernatant resulting from (iii) is centrifuged at a temperature of about 40° C. to about 50° C. (e.g., 41° C., 42° C., 43° C., 44° C., 45° C., 46° C., 47° C., 48° C., 49° C., or 50° C.). In some embodiments, in the second liquid-liquid centrifugation step, the demulsified supernatant resulting from (iii) is centrifuged at a temperature of about 40° C. In some embodiments, in the second liquid-liquid centrifugation step, the demulsified supernatant resulting from (iii) is centrifuged at a rate of from about 3,000 rpm to about 5,000 rpm (e.g., about 3,500 rpm to about 5,000 rpm, about 4,000 rpm to about 5,000 rpm, about 4,500 rpm to about 5,000 rpm, about 3,000 rpm to about 4,500 rpm, about 3,000 rpm to about 4,000 rpm, or about 3,000 rpm to about 3,500 rpm). In some embodiments, in the second liquid-liquid centrifugation step, the demulsified supernatant resulting from (iii) is centrifuged at a rate of about 4,100 rpm. In some embodiments, in the second liquid-liquid centrifugation step, the demulsified supernatant resulting from (iii) is centrifuged with a continuous centrifuge. In some embodiments, in the second liquid-liquid centrifugation step, the demulsified supernatant resulting from (iii) is centrifuged for from about 5 minutes to about 30 minutes (e.g., about 5 minutes to about 25 minutes, about 5 minutes to about 20 minutes, about 5 minutes to about 15 minutes, about 5 minutes to about 10 minutes, about 10 minutes to about 30 minutes, about 15 minutes to about 30 minutes, about 20 minutes to about 30 minutes, or about 25 minutes to about 30 minutes). In some embodiments, in the second liquid-liquid centrifugation step, the demulsified supernatant resulting from (iii) is centrifuged for about 15 minutes. In some embodiments, in a third liquid-liquid centrifugation step, the demulsified supernatant is centrifuged using a polishing centrifuge.

In some embodiments, between each of the liquid-liquid centrifugation steps, the oil component obtained from the liquid-liquid centrifugation is mixed with an aqueous solution that contains a base. In some embodiments, the base is a hydroxide salt. In some embodiments, the base is selected from NaOH, LiOH, KOH, and Ca(OH)₂. In some embodiments, the base is NaOH. In some embodiments, the concentration of hydroxide (OH⁻) in the aqueous solution is between about 0.0001 M and 10 M (e.g., 0.0001 M and about 0.8 M, about 0.0001 M and about 0.6 M, about 0.0001 M and about 0.4 M, about 0.0001 M and about 0.2 M, about 0.0001 M and about 0.01 M, about 0.001 M and about 10 M, about 0.01 M and about 10 M, about 0.1 M and about 10 M, about 2 M and about 10 M, about 4 M and about 10 M, about 6 M and about 10 M, or about 8 M and about 10 M). In some embodiments, the concentration of hydroxide (OH⁻) in the aqueous solution is about 1 M. In some embodiments, the concentration of hydroxide (OH⁻) in the aqueous solution is between about 0.0001 M and 1 M (e.g., 0.0001 M and about 0.1 M, about 0.0001 M and about 0.01 M, about 0.0001 M and about 0.001 M, about 0.001 M and about 1 M, about 0.01 M and about 1 M, or about 0.1 M and about 1 M). In some embodiments, the oil component is mixed with the aqueous solution that contains the base at a ratio of at least 1:0.5 (oil to aqueous solution, v/v). In some embodiments, the oil component is mixed with the aqueous solution that contains the base at a ratio of from about 0.5:1 to 1:0.1 (oil to aqueous solution, v/v). In some embodiments, the oil component is mixed with the aqueous solution that contains the base at a ratio of 1:1 (oil to aqueous solution, v/v).

In some embodiments, the oil component is mixed with the aqueous solution including the base at a temperature of from about 20° C. to about 80° C. (e.g., about 20° C. to about 70° C., about 20° C. to about 60° C., about 20° C. to about 50° C., about 20° C. to about 40° C., about 20° C. to about 30° C., about 30° C. to about 80° C., about 40° C. to about 80° C., about 50° C. to about 80° C., about 60° C. to about 80° C., or about 70° C. to about 80° C.). For example, the oil component may be mixed with the aqueous solution including the base at a temperature of about 40° C. In some embodiments, the oil component is mixed with the aqueous solution including the base for 0.25 hours or more. In some embodiments, the oil component is mixed with the aqueous solution including the base for 0.5 hours or more. In some embodiments, the oil component is mixed with the aqueous solution comprising the base for from about 0.1 hours to 10 hours (e.g., about 0.1 hours to about 9 hours, about 0.1 hours to about 8 hours, about 0.1 hours to about 7 hours, about 0.1 hours to about 6 hours, about 0.1 hours to about 5 hours, about 0.1 hours to about 4 hours, about 0.1 hours to about 3 hours, about 0.1 hours to about 2 hours, about 0.1 hours to about 1 hour, about 1 hour to about 10 hours, about 2 hours to about 10 hours, about 3 hours to about 10 hours, about 4 hours to about 10 hours, about 5 hours to about 10 hours, about 6 hours to about 10 hours, about 7 hours to about 10 hours, about 8 hours to about 10 hours, or about 9 hours to about 10 hours). In some embodiments, the oil component is mixed with the aqueous solution including the base for from about 0.5 hours to about 2 hours (e.g., about 0.5 hours to about 1.5 hours, about 0.5 hours to about 1 hour, about 1 hour to about 2 hours, or about 1.5 hours and about 2 hours). In some embodiments, the oil component is mixed with the aqueous solution including the base for about 1 hour.

In another aspect, the disclosure provides a method of purifying squalene from an extraction composition, wherein the extraction composition comprises squalene having previously been extracted from a squalene source. In some embodiments, the method includes (a) optionally evaporating the squalene from the extraction composition; and purifying the squalene resulting from (a) by way of chromatography.

In another aspect, the disclosure provides a method of purifying squalene from an extraction composition, wherein the method includes: (a) providing a composition comprising squalene having previously been extracted from a squalene source; and (c) optionally evaporating the squalene from (a); and (b) purifying the squalene resulting from (b) by way of chromatography.

In some embodiments, the squalene source is a fermentation source. In some embodiments, the fermentation source comprises yeast. In some embodiments, the squalene source is a plant source. In some embodiments the plant source comprises olive, soybean, grape seed, grape, hazelnut, peanut, corn, amaranth, rice, wheat germ, coriander, sesame, or sunflower. In some embodiments, the squalene source is an animal source. In some embodiments, the squalene source is a fungi source. In some embodiments, the fermentation source comprises a stramenopile source. In some embodiments, the stramenopile source comprises algae.

In an aspect, the disclosure provides a method of purifying squalene from an extraction composition, wherein the extraction composition comprises squalene having previously been extracted from a fermentation source that has been produced by culturing a population of host cells capable of producing squalene in a culture medium and under conditions suitable for the host cells to produce squalene including (a) optionally evaporating the squalene from the extraction composition; and (b) purifying the squalene resulting from (a) by way of chromatography.

In another aspect, the disclosure provides a method of purifying squalene from a fermentation composition including: (a) providing an extraction composition comprising squalene having previously been extracted from a fermentation source that has been produced by culturing a population of host cells capable of producing squalene in a culture medium and under conditions suitable for the host cells to produce squalene, the method comprising; (b) optionally evaporating the squalene from (a); and (c) purifying the squalene resulting from (b) by way of chromatography.

In some embodiments of the disclosure, the evaporation step includes fractional distillation, which may be used to isolate the squalene. In some embodiments of the disclosure, the evaporation step includes simple distillation, which may be used to isolate the squalene. In some embodiments, the squalene is evaporated by, e.g., initially heating the squalene to a temperature of from about 20° C. to about 90° C. (e.g., about 20° C. to about 90° C., about 20° C. to about 70° C., about 20° C. to about 50° C., about 20° C. to about 30° C., about 30° C. to about 90° C., about 50° C. to about 90° C., or about 70° C. to about 90° C.). In some embodiments, the squalene is evaporated by, e.g., initially heating the squalene to a temperature of from about 60° C. to about 70° C. (e.g., about 60° C., 61° C., 62° C., 63° C., 64° C., 65° C., 66° C., 67° C., 68° C., 69° C., or 70° C.). In some embodiments, the squalene is evaporated at a temperature of from about 150° C. to about 300° C. (e.g., about 150° C. to about 250° C., about 150° C. to about 275° C., about 150° C. to about 225° C., about 150° C. to about 200° C., about 150° C. to about 175° C., about 175° C. to about 300° C., about 200° C. to about 300° C., about 225° C. to about 300° C., about 250° C. to about 300° C., or about 275 to about 300° C.). In some embodiments, the squalene is evaporated at a temperature of from about 200° C. to about 280° C. (e.g., about 200° C., 205° C., 210° C., 215° C., 220° C., 225° C., 230° C., 235° C., 240° C., 245° C., 250° C., 255° C., 260° C., 265° C., 270° C., 275° C., or 280° C.). In some embodiments, the squalene is evaporated at a temperature of from about 200° C. to about 255° C. (e.g., about 200° C., 201° C., 202° C., 203° C., 204° C., 205° C., 206° C., 207° C., 208° C., 209° C., 210° C., 211° C., 212° C., 213° C., 214° C., 215° C., 216° C., 217° C., 218° C., 219° C., 220° C., 221° C., 222° C., 223° C., 224° C., 225° C., 226° C., 227° C., 228° C., 229° C., 230° C., 231° C., 232° C., 233° C., 234° C., 235° C., 236° C. 237° C., 238° C., 239° C., 240° C., 241° C., 242° C., 243° C., 244° C., 245° C., 246° C., 247° C., 248° C., 249° C., 250° C., 251° C., 252° C., 253° C., 254° C., or 255° C.). In some embodiments, the squalene is evaporated at a temperature of from about 200° C. to about 205° C. (e.g., about 200° C., 201° C., 202° C., 203° C., 204° C., or 205° C.).

In some embodiments, the squalene is evaporated under vacuum, such as at a pressure of about 0.5 to 5 torr (e.g., about 0.5 torr to about 1 torr, about 0.5 torr to about 2 torr, about 0.5 torr to about 3 torr, about 0.5 torr to about 4 torr, about 4 torr to about 5 torr, about 3 torr to about 5 torr, about 2 torr to about 5 torr, or about 1 torr to about 5 torr). In some embodiments, the squalene is evaporated under vacuum, such as at a pressure of about 0.7 torr to about 4 torr (e.g., about 0.7 torr to about 4 torr, 0.7 torr to about 3 torr, 0.7 to about 2 torr, about 2 torr to about 4 torr, or about 3 torr to about 4 torr). In some embodiments, the squalene is evaporated under vacuum, such as at a pressure of about 2 torr to about 4 torr (e.g., about 2 torr to about 3.5 torr, about 2 torr to about 3 torr, about 2 torr to about 2.5 torr, about 2.5 torr to about 4 torr, about 3 torr to about 4 torr, or about 3.5 torr to about 4 torr). In some embodiments, the squalene is evaporated under vacuum, such as at a pressure of about 0.7 torr to about 2 torr (e.g., about 0.7, 0.8, 0.9, 1, 1.2, 1.3, 1.4, 1.5, 1.6, 1.7 1.8, 1.9, or 2 torr). In some embodiments, the squalene is evaporated under vacuum, such as at a pressure of about 1 torr. In some embodiments, following the evaporating, the squalene is condensed and cooled to a temperature of about 70° C. or less. In some embodiments, following the evaporating, the squalene is condensed and cooled to a temperature of from about 20° C. to about 70° C. (e.g., about 20° C. to about 60° C., about 20° C. to about 50° C., about 20° C. to about 40° C., about 20° C. to about 30° C., about 30° C. to about 70° C., about 40° C. to about 70° C., about 50° C. to about 70° C., or about 60° C. to about 70° C.). In some embodiments, following the evaporating, the squalene is condensed and cooled to a temperature of from about 20° C. to about 25° C. (e.g., about 20° C., 21° C., 22° C., 23° C., 24° C., or 25° C.).

In some embodiments, the squalene is condensed and cooled under vacuum, optionally wherein the squalene is condensed and cooled at a pressure of about 1 torr. In some embodiments, the squalene is condensed and cooled under an inert gas. In some embodiments, the squalene is condensed and cooled under N₂. In some embodiments, the squalene is condensed and cooled under helium.

In some embodiments, the chromatography is performed by exposing the squalene to a polar resin and recovering the squalene from the resin. In some embodiments, the resin includes aluminum oxide. In some embodiments, the aluminum oxide is basic aluminum oxide. In some embodiments, the resin includes silica. In some embodiments, the aluminum oxide is acidic aluminum oxide. In some embodiments, the aluminum oxide is neutral aluminum oxide. In some embodiments, the resin has an average particle size of from about 50 μm to about 700 μm (e.g., about 50 μm to about 600 μm, about 50 μm to about 500 μm, about 50 μm to about 400 μm, about 50 μm to about 300 μm, about 50 μm to about 200 μm, about 50 μm to about 100 μm, about 100 μm to about 650 μm, about 200 μm to about 6500 μm, about 300 μm to about 650 μm, about 400 μm to about 650 μm, about 500 μm to about 650 μm, about 600 μm to about 650 μm, about 500 μm to about 700 μm, about 600 μm to about 700 μm, or about 300 μm to about 700 μm). In some embodiments, the resin has an average particle size of from about 50 μm to about 250 μm (e.g., about 50 μm to about 150 μm, about 50 μm to about 100 μm, about 50 μm to about 75 μm, about 75 μm to about 200 μm, about 100 μm to about 200 μm, about 125 μm to about 200 μm, about 150 μm to about 200 μm, about 50 μm to about 225 μm, or about 200 μm to about 250 μm). In some embodiments, the resin has an average particle size of from about 300 μm to 650 μm (e.g., about 300 μm to 600 μm, about 300 μm to about 500 μm, about 300 μm to about 400 μm, about 400 μm to about 650 μm, about 500 μm to about 650 μm, or about 600 μm to about 650 μm). In some embodiments, the resin requires an activation step; for example, the activation step may be a drying step. In some embodiments, the chromatography is performed at ambient temperature. In some embodiments, the chromatography is performed using a flow rate of from about 0.2 bed volumes per hour (BV/hr) to about 5 BV/hr (e.g., about 0.2 BV/hr to about 4 BV/hr, 0.2 BV/hr to about 3 BV/hr, about 0.2 BV/hr to about 2 BV/hr, about 2 BV/hr to about 5 BV/hr, about 3 to about 5 BV/hr, about 4 BV/hr to about 5 BV/hr, about 0.5 BV/hr to about 2 BV/hr, about 0.2 BV/hr to about 1 BV/hr, or about 0.5 BV/hr to about 1 BV/hr). In some embodiments, the chromatography is performed using a flow rate of from about 1.5 BV/hr to about 3 BV/hr (e.g., about 1.5 BV/hr to about 2.5 BV/hr, 1.5 BV/hr to about 2 BV/hr, about 2 BV/hr to about 3 BV/hr, or about 2.5 BV/hr to about 3 BV/hr). In some embodiments, the chromatography is performed using a flow rate of from about 2 BV/hr to about 2.5 BV/hr (e.g., about 2 BV/hr, 2.1 BV/hr, 2.2 BV/hr, 2.3 BV/hr, 2.4 BV/hr, or 2.5 BV/hr).

In some embodiments, an antioxidant is added to the squalene. In some embodiments, the antioxidant is Vitamin E. In some embodiments, the Vitamin E is present at a concentration ranging from about 100 to 1000 ppm (e.g., about 100 ppm to about 900 ppm, about 100 ppm to about 700 ppm, about 100 ppm to about 500 ppm, about 100 ppm to about 300 ppm, about 300 ppm to about 1000 ppm, about 500 ppm to about 1000 ppm, about 700 ppm to about 1000 ppm, about 900 ppm to about 1000 ppm, or about 300 ppm to about 800 ppm). In some embodiments, the Vitamin E is present at a concentration of about 500 ppm.

In some embodiments, the host cell is a yeast cell. In some embodiments, the yeast cell is S. cerevisiae.

In some embodiments, the squalene is isolated from the fermentation composition with a purity of from about 90% (w/w) to about 100% (w/w) (e.g., at least: 90% (w/w), 91% (w/w), 92% (w/w), 93% (w/w), 94% (w/w), 95% (w/w), 96% (w/w), 97% (w/w), 98% (w/w), 99% (w/w), 99.9% (w/w), or 100% (w/w)). In some embodiments, the squalene is isolated from the fermentation composition with a purity of from about 95% (w/w) to about 100% (w/w) (e.g., at least: 96% (w/w), 97% (w/w), 98% (w/w), 99% (w/w), 99.9%, or 100% (w/w)). In some embodiments, the squalene is isolated from the fermentation composition with a purity of from about 99.5% (w/w) to about 99.9% (w/w) (e.g., at least: about 99.5% (w/w), 99.6% (w/w), 99.7% (w/w), 99.8% (w/w), 99.9% (w/w), or 100% (w/w)).

In another aspect, the disclosure provides a composition including squalene, wherein the composition is produced by any one of the methods described herein. In some embodiments, the squalene has a purity of from about 90% (w/w) to about 100% (w/w) (e.g., at least: about 90% (w/w), 91% (w/w), 92% (w/w), 93% (w/w), 94% (w/w), 95% (w/w), 96% (w/w), 97% (w/w), 98% (w/w), 99% (w/w), 99.9% (w/w), 100% (w/w)). In some embodiments, the squalene has a purity of from about 95% (w/w) to about 100% (w/w) (e.g., at least: about 96% (w/w), 97% (w/w), 98% (w/w), 99% (w/w), or 99.9% (w/w)). In some embodiments, the squalene has a purity of from about 99.5% (w/w) to about 99.9% (w/w) (e.g., at least: about 99.6% (w/w), 99.7% (w/w), 99.8% (w/w), 99.9% (w/w), or 100% (w/w)).

In another aspect, the disclosure provides a pharmaceutical composition including squalene and one or more pharmaceutically acceptable carriers, diluents, or excipients. The purity of the squalene may be, for example, from about 99.5% (w/w) to about 100% (w/w) (e.g., at least: 99.5% (w/w), 99.6% (w/w), 99.7% (w/w), 99.8% (w/w), 99.9% (w/w), or 100% (w/w)). In some embodiments, the squalene is present with one or more impurities, and wherein the one or more impurities are present in a concentration of about 0.5% (w/w) or less. In some embodiments, the one or more impurities are present in a concentration of about 0.4% (w/w) or less, optionally wherein the one or more impurities are present in a concentration of about 0.3% (w/w) or less, optionally wherein the one or more impurities are present in a concentration of about 0.2% (w/w) or less, optionally wherein the one or more impurities are present in a concentration of about 0.1% (w/w) or less. In some embodiments, the one or more impurities include a fatty acid and/or a sterol.

In another aspect, the disclosure provides an adjuvant formulation including squalene produced any one of the methods described herein and a pharmaceutically acceptable carrier, diluent, or excipient.

In another aspect, the disclosure provides an adjuvant formulation including any one of the compositions described herein.

In another aspect, the disclosure provides a vaccine including a therapeutically or prophylactically effective amount of the adjuvant formulation any one of the antigens described herein.

In another aspect, the disclosure provides a vaccine including the squalene produced by any one of the methods described herein and an antigen. In some embodiments, the antigen is a protein expressed by a virus. In some embodiments, the antigen is encoded by a nucleic acid molecule encoding a protein expressed by a virus. In some embodiments, the nucleic acid molecule is a deoxyribonucleic acid (DNA) or a ribonucleic acid (RNA) molecule. In some embodiments, the virus is selected from influenza virus, hepatitis A virus, hepatitis B virus, hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus, rhinovirus, mumps virus, poliovirus, human enterovirus, coxsackievirus, human papilloma virus, adeno-associated virus, astrovirus, JC virus, BK virus, SV40 virus, Norwalk virus, rotavirus, human immunodeficiency virus (HIV), human T-lymphotropic virus, SARS-COV-2, MERS-COV, SARS-COV, OC43, and HKU1.

In some embodiments, the antigen is a protein expressed by a bacterium. In some embodiments, the antigen is encoded by a nucleic acid molecule encoding a protein expressed by a bacterium. In some embodiments, the nucleic acid molecule is a DNA or RNA molecule. In some embodiments, the bacterium belongs to a genus selected from Mycobacterium, Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus. In some embodiments, the antigen is a protein expressed by a parasite. In some embodiments, the antigen is encoded by a nucleic acid molecule encoding a protein expressed by a parasite. In some embodiments, the nucleic acid molecule is a DNA or RNA molecule. In some embodiments, the parasite is selected from Plasmodium malariae, Plasmodium vivax, Plasmodium ovale, Plasmodium falciparum, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Trichomonas vaginalis, and Histomonas meleagridis, Richuris trichiura, Ascaris lumbricoides, Enterobius vermicularis, Ancylostoma duodenale, Necator americanus, Strongyloides stercoralis, Wuchereria bancrofti, Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus.

In some embodiments, the antigen is a protein expressed by a cancer cell. In some embodiments, the antigen is encoded by a nucleic acid molecule encoding a protein expressed by a cancer cell. In some embodiments, the nucleic acid molecule is a DNA or RNA molecule. In some embodiments, the protein is selected from gp100, Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-1, MUC-16, L1CAM, HERV-K-MEL, KK-LC-1, KM-HN-1, LAGE-1, Sp17, TAG-1, TAG-2, ENAH (hMena), mammaglobin-A, NY-BR-1, BAGE-1, MAGE-A1, MAGE-A2, MAGE-A4, MAGE-A6, MAGE-A9, MAGE-A10, MAGE-A12, MAGE-C2, mucink, SSX-2, SSX-4, TRAG-3, c-myc, cyclin B1, p62, Survivin, CD45, DKK1, RU2AS, Telomerase, K-ras, G250, Hepsin, Intestinal carboxyl esterase, α-foetoprotein, M-CSF, PSMA, CASP-5, COA-1, OGT, OS-9, TGF-βRII, gp70, CALCA, CD274, mdm-2, α-actinin-4, Elongation factor 2, ME1, NFYC, GAGE-1/2/8, GAGE-3/4/5/6/7, XAGE-1b/GAGED2a, STEAP1, PAP, PSA, FGF5, hsp70-2, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, GnTVf, LY6K, and NA88-A.

In another aspect, the disclosure provides a method of inducing an immune response (e.g., an antigen-specific immune response) in a subject, the method including administering to the subject any of the vaccines described herein. In some embodiments, the subject is a mammal, such as a human.

### Definitions

As used in this specification and the appended claims, the singular forms “a,” “an,” and “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein, the term “antigen” refers to a molecule or a portion of a molecule capable of being bound by a selective binding agent, such as an antibody, and additionally capable of being used in an animal to produce antibodies capable of binding to an epitope of each antigen. An antigen may have one or more epitopes.

As used herein, the term “capable of producing” refers to a host cell which includes the enzymes necessary for the production of a given compound in accordance with a biochemical pathway that produces the compound. For example, a cell (e.g., a yeast cell) that is “capable of producing” squalene is one that contains the enzymes necessary for production of the squalene according to the squalene biosynthetic pathway.

As used herein, the term “derived from” in the context of an antigen (e.g., a pathogen antigen or a cancer cell antigen described herein) refers to a molecular substance (e.g., a protein, carbohydrate, or nucleic acid, such as DNA or RNA) that is characteristic of the antigen. An antigen that is “derived from” a pathogen (e.g., a virus, bacterium, or protozoan) or a cancer cell may be, for example, a protein, peptide fragment, or carbohydrate that is naturally expressed by the pathogen (e.g., the virus, bacterium, or protozoan) or cancer cell. Alternatively, the antigen may be a nucleic acid component of the pathogen (e.g., the virus, bacterium, or protozoan) or cancer cell, such as a DNA or RNA molecule that encodes all or a part of a pathogen protein (e.g., a viral, bacterial, or protozoan protein, such as a coat protein or cell wall protein) or a cancer cell protein. Synthetic antigens derived from or based upon a pathogen or cancer cell nucleic acid or protein sequence (e.g., a viral, bacterial, or protozoan component (for example, coat protein)) are also included in the invention. Additional examples of antigens that are “derived from” a pathogen (e.g., a virus, bacterium, or protozoan) or cancer cell include proteins or peptide fragments thereof that result from the processing of the pathogen by the immune system of a subject (e.g., a mammalian subject, such as a human). Antigens of this type include, without limitation, peptide fragments that associate with major histocompatibility complex (MHC) proteins of an antigen-presenting immune cell upon processing of a pathogen by the immune system of a subject (e.g., a mammalian subject, such as a human). Examples of antigen-presenting immune cells include, without limitation, macrophages and dendritic cells.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “evaporation” describes a process by which at least a portion of a liquid undergoes a state change to have a gaseous state. For example, evaporation may be used to separate two liquids from one another or to remove one liquid from a mixture containing one or more additional liquids. In some embodiments, evaporation includes the process of distillation, in which a liquid not only changes phase to a gaseous state, but is subsequently condensed back to a liquid form. In some embodiments of the disclosure, a distillation is performed by heating a mixture of liquids such that a lower-boiling point substance begins to evaporate, changing phase from a liquid to a gaseous state, while leaving the remaining liquid(s) in the mixture in a liquid phase. The lower-boiling point substance may then be condensed, e.g., upon exposure to reduced temperature, thereby: (1) returning the lower-boiling point substance to a liquid phase, and (2) separating the lower-boiling point substance from the remaining liquid(s) in the mixture.

The distillation may be, for example, a “simple distillation,” which refers to a process in which a mixture of liquids having substantially different boiling points (e.g., boiling points that differ from one another by about 25 C.° or more) is separated by heating the mixture until substantially all of the lower-boiling point substance evaporates and substantially all of the higher-boiling point substance remains in the liquid phase. The vapor of the lower-boiling point substance, in turn, is condensed so as to return the lower-boiling point substance to the liquid phase. In some embodiments, the distillation may be a “fractional distillation,” a process that is used, e.g., for separating highly miscible liquids and/or those having boiling points that differ by less than about 25 C°. A fractional distillation process involves heating a mixture containing the liquids such that the resulting vapor enters a fractionating column. The fractionating column is a column (e.g., a vertical, inclined, or horizontal column) configured so as to have a temperature gradient: the bottom of the fractionating column (i.e., the point at which the vapor enters the column) is the warmest, and the top of the fractionating column is the coolest. As the vapor proceeds upward through the column, the vapor is enriched for the lower-boiling point substance as a result of the temperature gradient. Once enriched for the lower-boiling point substance, the vapor exits the fractionating column and is exposed to a reduced temperature, thereby condensing the lower-boiling point substance and resolving the liquid mixture into its components.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted therefrom.

As used herein, the term “extraction composition” refers to a composition containing a desired biosynthetic product (e.g., squalene) that has been obtained by way of a previously conducted extraction of the product from a mixture (e.g., a mixture containing the product and one or more impurities, such as a mixture derived from a biological system that is capable of biosynthetically producing the product). For example, as used herein, an “extraction composition” may contain squalene that has been previously extracted from a squalene source. Exemplary squalene sources that may be used in conjunction with the compositions and methods of the disclosure include, without limitation, fermentation sources, plant sources, animal sources, and stramenopile sources.

As used herein, the term “fermentation composition” refers to a composition which contains host cells and products or metabolites produced by the genetically modified host cells. An example of a fermentation composition is a whole cell broth, which may be the entire contents of a vessel, including cells, aqueous phase, and compounds produced from the host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refers to a set of at least two different coding sequences, where the coding sequences encode enzymes that catalyze different parts of a synthetic pathway to form a desired product (e.g., squalene). In a genetic pathway, a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “heterologous” refers to what is not normally found in nature. The term “heterologous nucleic acid” refers to a nucleic acid not normally found in a given cell in nature. A heterologous nucleic acid can be: (a) foreign to its host cell, i.e., exogenous to the host cell such that a host cell does not naturally contain the nucleic acid; (b) naturally found in the host cell, i.e., endogenous or native to the host cell, but present at an unnatural quantity in the cell (i.e., greater or lesser quantity than naturally found in the host cell); (c) be naturally found in the host cell but positioned outside of its natural locus. A “heterologous” polypeptide refers to a polypeptide that is encoded by a “heterologous nucleic acid”. Thus, for example, a “heterologous” polypeptide may be naturally produced by a host cell but is encoded by a heterologous nucleic acid that has been introduced into the host cell by genetic engineering. For example, a “heterologous” polypeptide can include embodiments in which an endogenous polypeptide is produced by an expression construct and is overexpressed in the host cell compared to native levels of the polypeptide produced by the host cell.

As used herein, the term “medium” refers to culture medium and/or fermentation medium. The term “host cell” as used in the context of this disclosure refers to a microorganism, such as yeast, and includes an individual cell or cell culture including a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion.

As used herein, the term “pharmaceutical composition” refers to a mixture containing a therapeutic compound or prophylactic compound to be administered to a subject, such as a mammal, e.g., a human, in order to prevent, treat or control a particular disease or condition affecting or that may affect the mammal.

As used herein, the term “pharmaceutically acceptable” refers to those compounds, materials, compositions and/or dosage forms which are suitable for contact with the tissues of a subject, such as a mammal (e.g., a human) without excessive toxicity, irritation, allergic response, or other deleterious complications commensurate with a reasonable benefit/risk ratio.

As used herein, the term “pharmaceutically acceptable salt” means any pharmaceutically acceptable salt of a compound described herein. For example, pharmaceutically acceptable salts of any of the compounds described herein include those that are within the scope of sound medical judgment, suitable for use in contact with the tissues of humans and animals without undue toxicity, irritation, or allergic response, and are commensurate with a reasonable benefit/risk ratio. Examples of pharmaceutically acceptable salts are described in: Berge et al., J. Pharmaceutical Sciences 66:1-19, 1977 and in Pharmaceutical Salts: Properties, Selection, and Use, (Eds. P. H. Stahl and C.G. Wermuth), Wiley-VCH, 2008. Such salts can be prepared, for example, in situ during the final isolation and purification of a compound described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. The compounds may be prepared or used as pharmaceutically acceptable salts synthesized as addition products of pharmaceutically acceptable acids or bases. Suitable pharmaceutically acceptable salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

As used herein, the term “production” generally refers to an amount of compound produced by a host cell provided herein. In some embodiments, production is expressed as a yield of the compound by the host cell. In other embodiments, production is expressed as a productivity of the host cell in producing the compound.

The terms “modified,” “recombinant” and “engineered,” when used to modify a host cell described herein, refer to host cells or organisms that do not exist in nature, or express compounds, nucleic acids or proteins at levels that are not expressed by naturally occurring cells or organisms.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and/or cells) isolated from a subject.

As used herein, the term “squalene source” refers to a system, such as a biological system (e.g., one or more cells, single-celled organisms, or multi-celled organisms) that is capable of producing squalene. A squalene source may be naturally capable of producing squalene or may be modified in such a way so as to be capable of making squalene. A squalene source may be modified to be capable of producing squalene by a genetic modification that causes the squalene source to express one or more enzymes of a squalene biosynthetic pathway. Examples of squalene sources include but are not limited to plants, animals, stramenopiles, host cells, or fermentation compositions.

As used herein, the terms “subject” and “patient” are interchangeable and refer to an organism that receives therapeutic or prophylactic treatment for a particular disease or condition as described herein. Examples of subjects and patients include mammals, such as humans.

As used herein, “vaccine” refers to a formulation which induces a protective immune response sufficient to induce immunity to prevent and/or ameliorate an infection or disease and/or to reduce at least one symptom of an infection or disease and/or to enhance the efficacy of another dose of the synthetic nanoparticle. Typically, the vaccine includes a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the production of antibodies and/or cytokines and/or the activation of cytotoxic T cells, antigen presenting cells, helper T cells, dendritic cells and/or other cellular responses.

### BRIEF DESCRIPTION OF THE DRAWINGS

The application file contains at least one drawing executed in color. Copies of this patent or patent application with color drawings will be provided by the Office upon request and payment of the necessary fee.

FIG. 1 is a schematic showing exemplary steps performed for extraction and purification of squalene from a fermentation composition using compositions and methods of the disclosure.

FIG. 2 is a photograph showing the fermentation composition resulting from exposure to a surfactant at various concentrations, in accordance with the protocol described in Example 1, below.

FIG. 3 shows a series of photographs of representative samples of liquid-solid light phase (left-most) processed through demulsification to the liquid-liquid (L/L) feed (middle) and centrifuged to generate crude oil and aqueous streams (right-most), in accordance with the protocol described in Example 1, below.

FIG. 4 is a graph showing a simulation distillation profile from crude oil produced in the pilot plant (blue), wiped film evaporator (WFE) distillate generated from the crude oil, and Sigma Standard Squalene (>98% wt). Inset plot is a zoomed in plot of the baseline.

FIG. 5 is a graph showing the effect of temperature on single-stage evaporation yield (right ordinate) and distillate purity (left ordinate).

FIG. 6 is a graph showing the relationship between evaporation yield (orange), distillate purity (blue), and distillate-to-feed ratio. Data from the 2″ WFE are shown with circles and data from the Kugelrohr are shown with diamonds. Mass balance modelling shows dependence for two scenarios: (1) 82% wt squalene feed and 96% wt squalene distillate (shown with solid lines) and (2) 77% wt squalene feed and 99% wt squalene distillate (shown with dotted line).

FIG. 7 is a graph showing the squalene area purity as a function of bed volume of effluent collected, which shows a decrease in area percentage after passing 9 bed volumes (BV) of feed through the column. Area percent at BV=0 is of the feed.

FIG. 8 shows the GC-MS chromatograms of the crude oil (before base washing) and the base-washed crude oil depicting the impurity profile of fatty acids (residence times from 11-15 min) and sterols (residence times from 17.5-19 min), as identified on the image.

FIG. 9 is a graph showing the average distillate squalene concentration as measured by area % by temperature (C).

FIG. 10 shows the distillate purity, [Squalene] (Area %, light gray circles, left ordinate) and distillate to feed ratio (D:F) (g/g, dark gray circles, right ordinate) binned by evaporation temperature and plotted against pressure. Error bars represent standard error of the mean.

FIG. 11 is a bar showing the average distillate impurity concentration (Area %) plotted as stacked bars with light impurities (dark gray bars) stacked on top of heavy impurities (light gray bars).

FIG. 12 shows a bubble plot of the relative area % profile of non-squalene peaks detected in distillate produced at 200° C. by their relative retention time (RRT) to the main squalene peak.

FIG. 13 shows a bubble plot of the relative area % profile of non-squalene peaks detected in distillate produced at 225° C. by their relative retention time (RRT) to the main squalene peak.

FIG. 14 shows a bubble plot of the relative area % profile of non-squalene peaks detected in distillate produced at 250° C. by their relative retention time (RRT) to the main squalene peak.

FIG. 15 is a graph showing the average percent yield (light gray circles, left ordinate) and D:F (dark gray circles, right ordinate) achieved using various evaporation temperatures and pressures during distillation.

FIG. 16 is a graph showing the average yield by temperature for operating pressures of 0.70 torr, 1 torr, and 2 torr during distillation.

FIG. 17 shows a pair of graphs showing the impurities present in distillate distilled at 250° C. and 255° C.

FIG. 18 is a graph showing the impurities in distillate distilled at 250° C. following alumina polishing to 12 bed volumes.

FIG. 19 is a graph showing the impurities in distillate distilled at 255° C. following alumina polishing to 12 bed volumes.

FIG. 20 is a graph showing the impurities in distillate distilled at 250° C. following alumina polishing.

FIG. 21 is a graph showing the predicted impurities in distillate distilled at 255° C. following alumina polishing.

FIG. 22 is a graph showing the prediction of impurities present in distillates distilled at 250° C. or 255° C. purified with various flow rates

### DETAILED DESCRIPTION

The present disclosure provides methods of isolating squalene such that the resulting squalene is highly pure. The squalene may be isolated from any source such as a fermentation composition, a plant, or animal (e.g., fish liver oil). Given the interest in producing and isolating squalene from sustainable sources, the challenge of purifying squalene with high purity has been significant. Since squalene produced biosynthetically is sequestered intracellularly and is not secreted, homogenization is used to release squalene from the host cell interior into the surrounding extracellular media. However, homogenization also releases a complex matrix of impurities that are strikingly difficult to separate from the desired squalene product.

It has presently been discovered that combining a unique series of evaporation, and chromatography steps results in squalene compositions having remarkable levels of purity (e.g., levels of purity of 99% (w/w) and above). For example, using the methods and compositions described herein, squalene may be purified from a squalene source, (e.g., a plant, animal or fermentation composition). In some instances, the fermentation composition may be produced by culturing host cells capable of synthesizing squalene in a culture medium and under conditions suitable for the host cells to produce squalene. To recover squalene from the fermentation composition, the fermentation composition may be subject to one or more extraction steps, each of which may include homogenizing the fermentation composition, separating the homogenized fermentation into sediment and supernatant by centrifugation, demulsifying the supernatant, and separating the demulsified supernatant into an aqueous component and an oil component. The squalene extracted from a squalene source may be evaporated (e.g., distilled) and purified by way of chromatography so as to reach a form that is substantially free of cellular impurities.

The sections that follow provide a description of exemplary compositions and methods that may be used to perform the fermentation, extraction, evaporation, and chromatography steps of the disclosure.

### Biosynthetic Production of Squalene

There are two primary isoprenoid pathways used for the production of squalene in host cells. Host cells that may be used in conjunction with the compositions and methods disclosed herein may express the enzymes involved in either of these pathways. For example, squalene can be synthesized by way of the mevalonate (MVA) biosynthetic pathway. Alternatively, squalene can be synthesized by way of the 2-C-methyl-D-erythritol 4-phosphate (MEP) pathway (this latter pathway is also referred to as the DXP (1-deoxy-D-xylulose-5-phosphate) pathway, depending upon the organism in which the biosynthesis occurs). Prokaryotes, such as bacteria and cyanobacteria, often utilize the MEP pathway, whereas eukaryotes, including yeast, higher fungi, plants, and animals, utilize the MVA pathway for the biosynthesis of squalene.

The MVA pathway starts with the condensation of three units of acetyl-CoA to form 3-hydroxy-3-methylglutaryl-CoA (HMG-COA) via acetoacetyl-CoA, in consecutive reactions catalyzed by the enzymes acetoacetyl-CoA thiolase (AACT) and HMG-COA synthase (HMGS). HMG-COA is subsequently reduced to MVA in presence of cofactor NADPH by HMG-COA reductase (HMGR). MVA is then phosphorylated by the enzymes MVA kinase and phospho-MVA kinase, thereby producing MVA-5-diphosphate. Subsequently, decarboxylation of MVA-5-diphosphate takes place in the presence of adenosine triphosphate (ATP) to form isopentyl diphosphate (IPP). Next, IPP interconverts into dimethylallyl diphosphate (DMAPP) through a reaction catalyzed by IPP isomerase (IDI). Condensation of both molecules, IPP and DMAPP, by farnesyl diphosphate synthase (FPS) results in geranyl diphosphate (GPP), which is followed by a condensation reaction converting GPP to a 15-carbon isopentyl block unit-FPP (farnesyl pyrophosphate). Eventually, two molecules of FPP are used to synthesize squalene by an NADPH mediated reaction catalyzed by squalene synthase (SQS, encoded by ERG9). The pathway then continues onward to synthesize sterols.

The MEP pathway begins with the condensation of glyceraldehyde-3-phosphate (GA3P) and pyruvate to build DXP that is catalyzed by DXP synthase (DXS). DXP then undergoes reduction to form MEP by DXP reductoisomerase (DXR) or its isozyme DRL (DXR-like). MEP is subsequently transformed into IPP and DMAPP in the subsequent steps by a series of enzymes. The remaining steps from IPP to FPP are identical to the MVA pathway.

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing squalene provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag Gmbh & Co. KDaA, Weinheim, Germany.

In some embodiments, the culture medium is any culture medium in which a microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

### Host Cell Strains

In some embodiments of the present disclosure, the host cell is a yeast cell. Yeast cells useful in conjunction with the compositions and methods described herein include yeast that have been deposited with microorganism depositories (e.g. IFO, ATCC, etc.), such as those that belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellesis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In some embodiments, the host cell is Saccharomyces cerevisiae. Saccharomyces cerevisiae strains suitable for cultivation to produce squalene as disclosed herein include, but are not limited to, Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, CEN.PK, CEN.PK2, and AL-1. In some embodiments, the host cell is a strain of Saccharomyces cerevisiae selected from the group consisting of PE-2, CAT-1, VR-1, BG-1, CR-1, and SA-1. In certain aspects, the strain of Saccharomyces cerevisiae is PE-2. In certain embodiments, the strain of Saccharomyces cerevisiae is CAT-1. In some aspects, the strain of Saccharomyces cerevisiae is BG-1.

In some embodiments, the host cell is Kluyveromyces marxianus. Kluyveromyces marxianus can provide several advantages for industrial production, including high temperature tolerance, acid tolerance, native uptake of lactose, and rapid growth rate. Beneficially, this yeast has sufficient genetic similarity to Saccharomyces cerevisiae such that similar or identical promoters and codon optimized genes can be used among the two yeast species.

### Other Squalene Sources

Squalene may be isolated from a naturally occurring source or a non-naturally occurring source. In some embodiments, the squalene source used in the disclosure is from a plant, animal, or fermentation composition.

In some embodiments, the squalene source is an animal. In some embodiments, the animal is a vertebrate. In some embodiments, the animal source is a fish. For example, the fish may be salmon. In some embodiments, the animal source is a shark. The greatest concentration of squalene in the living world is met in the liver of certain species of fish, especially sharks living in sea at depth under 400 m. In the case of deep-sea sharks, the liver is the main organ for lipids' storage, being in the same time an energy source and means for adjusting the buoyancy. In their case, the unsaponifiable matter represents 50-80% of the liver, the great majority thereof being squalene.

In some embodiments, the squalene source used in the disclosure may be a plant source. Squalene has been identified in many plant oils in different concentrations. Squalene has been found to be in olive oil, soybean oil, grape seed oil, hazelnut oil, peanut oil, sesame oil, sunflower oil, coriander seed oil, and in corn oil. Squalene may also be found in Amaranthus, rice bran, wheat germ, Monkey Jack, and olives.

In some embodiments, the squalene source is a stramenopile. In some embodiments, the squalene source is algae. In some embodiments, the squalene source is from green algae. For example, the stramenopile may be B. bruenii, Auranthiochytrium, or C. reinhardtii.

In some embodiments, the squalene source is a fermentation source. In some embodiments, the fermentation source comprises yeast. For example, the fermentation source may include K. lactis, S. cerevisiae, or Pseudozyma sp.

### Methods of Isolating Squalene

Described herein are methods of purifying squalene, methods of isolating squalene from a squalene source, as well as methods of making squalene having high purity. The methods described herein may include, for example, in some cases extracting squalene from a fermentation composition, evaporating (e.g., distilling) the extracted squalene, and purifying the evaporated (e.g., distilled) squalene to recover squalene product in high purity. Exemplary potential extraction, evaporation, and chromatography steps are described in further detail in the sections that follow.

### Homogenization

Using the compositions and methods described herein, squalene may be extracted from a squalene source, for example, a fermentation composition that has been produced by culturing a population of host cells capable of producing squalene in a culture medium. The extraction may be performed by homogenizing the fermentation composition. The fermentation composition may be homogenized in one or more steps. For example, the fermentation composition may be homogenized in from one to five steps (e.g., one step, two steps, three steps, four steps, or five steps). In particular embodiments, the fermentation composition is homogenized in two steps.

Homogenizing the fermentation composition may be carried out at high pressure. In some embodiments, homogenization of the fermentation composition is performed at a pressure of about 400 bar to about 1,200 bar (e.g., between 400 bar and 900 bar, 400 bar and 800 bar, 400 bar and 700 bar, 400 bar and 600 bar, 400 bar and 500 bar, 500 bar and 1000 bar, 600 bar and 1000 bar, 700 bar and 1000 bar, 800 bar and 1000 bar, or between 900 bar and 1000 bar). For example, each step may include homogenizing the fermentation composition at a pressure of between about 800 bar and about 1,000 bar (e.g., about 810 bar, 820 bar, 830 bar, 840 bar, 850 bar, 860 bar, 870 bar, 880 bar, 890 bar, 900 bar, 910 bar, 920 bar, 930 bar, 940 bar, 950 bar, 960 bar, 970 bar, 980 bar, 990 bar, or 1,000 bar). In particular embodiments, each step includes homogenizing the fermentation composition at a pressure of about 900 bar. Prior to homogenizing the fermentation composition, the fermentation composition may be diluted in water.

In some embodiments, following homogenization, the fermentation composition is diluted in water, for example, to a final concentration of from about 20% to about 40% solid material (v/v) (e.g., about 20%, 21%, 22%, 23%, 24%, 25%, 26%, 27%, 28%, 29%, 30%, 40%, 41%, 42%, 43%, 44%, 45%, 46%, 47%, 48%, 49%, or 50% solid material (v/v)). For example, the fermentation composition may be diluted such that the diluted fermentation composition is from about 30% to about 35% solid material (v/v) (e.g., about 30%, 31%, 32%, 33%, or 35% solid material (v/v)).

### Centrifugation

Extraction of the squalene from a squalene source of the disclosure may include centrifugation. The centrifugation step may include solid-liquid centrifugation and/or liquid-liquid centrifugation.

For example, solid-liquid centrifugation may be used to separate a squalene source, for example, a homogenized fermentation composition, into sediment and a supernatant. Prior to separating the homogenized fermentation composition into sediment and supernatant by way of solid-liquid centrifugation, the homogenized fermentation composition may be heated to a temperature of from about 18° C. and about 75° C. (e.g., about 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or 75° C.). Additionally or alternatively, prior to solid-liquid centrifugation, the homogenized fermentation composition may be diluted with water to a final concentration of from about 20% to about 30% solid material (v/v) (e.g., about 20%, 21%, 22%, 23%, 24%, 25%, 26%, 27%, 28%, 29%, or 30% solid material (v/v)). In some embodiments, the homogenized fermentation composition may be diluted with water to a final concentration of about 25% solid material (v/v).

The solid-liquid centrifugation may be performed at rate of from about 3,000 revolutions per minute (rpm) to about 5,000 rpm (e.g., about 3,500 rpm to about 5,000 rpm, about 4,000 rpm to about 5,000 rpm, about 4,500 rpm to about 5,000 rpm, about 3,000 rpm to about 4,500 rpm, about 3,000 rpm to about 4,000 rpm, or about 3,000 rpm to about 3,500 rpm). In some embodiments, the solid-liquid centrifugation is performed at rate of from 4,1000 rpm. Furthermore, the solid-liquid centrifugation may be performed for between about 5 minutes and about 30 minutes (e.g., about 5 minutes to about 25 minutes, about 5 minutes to about 20 minutes, about 5 minutes to about 15 minutes, about 5 minutes to about 10 minutes, about 10 minutes to about 30 minutes, about 15 minutes to about 30 minutes, about 20 minutes to about 30 minutes, or about 25 minutes to about 30 minutes). For example, the homogenized fermentation composition may be centrifuged for about 15 minutes.

Liquid-liquid centrifugation may be used to separate an aqueous component and an oil component. For example, liquid-liquid centrifugation may be used to separate a supernatant (e.g., following demulsification) into an aqueous component and an oil component. The liquid-liquid centrifugation may be performed in one or more steps. For instance, the liquid-liquid centrifugation may be performed in two steps. In a first liquid-liquid centrifugation step, the demulsified supernatant may be centrifuged at a temperature of from about 50° C. to about 90° C. (e.g., 50° C. to about 80° C., about 50° C. to about 70° C., about 50° C. to about 60° C., about 60° C. to about 90° C., about 70° C. to about 90° C., or about 80° C. to about 90° C.). For example, in the first liquid-liquid centrifugation step, the demulsified supernatant may be centrifuged at a temperature of about 70° C. In the first liquid-liquid centrifugation step, the demulsified supernatant may be centrifuged at a rate of from about 3,000 rpm to about 5,000 rpm (e.g., about 3,500 rpm to about 5,000 rpm, about 4,000 rpm to about 5,000 rpm, about 4,500 rpm to about 5,000 rpm, about 3,000 rpm to about 4,500 rpm, about 3,000 rpm to about 4,000 rpm, or about 3,000 rpm to about 3,500 rpm). For example, the demulsified supernatant may undergo a first liquid-liquid centrifugation step at a rate of about 4,100 rpm. The first liquid-liquid centrifugation step of the demulsified supernatant may be performed for about 5 minutes to about 30 minutes (e.g., about 5 minutes to about 25 minutes, about 5 minutes to about 20 minutes, about 5 minutes to about 15 minutes, about 5 minutes to about 10 minutes, about 10 minutes to about 30 minutes, about 15 minutes to about 30 minutes, about 20 minutes to about 30 minutes, or about 25 minutes to about 30 minutes). For example, the first liquid-liquid centrifugation may be performed for about 15 minutes.

The demulsified supernatant may undergo a second liquid-liquid centrifugation step at a temperature of from about 25° C. to about 50° C. (e.g., about 25° C. to about 60° C., about 25° C. to about 50° C., about 25° C. to about 40° C., about 25° C. to about 30° C., about 30° C. to about 70° C., about 40° C. to about 70° C., or about 50° C. to about 70° C.)). For example, the second liquid-liquid centrifugation step may be performed at temperature of between about 40° C. and 50° C. (e.g., 41° C., 42° C., 43° C., 44° C., 45° C., 46° C., 47° C., 48° C., 49° C., or 50° C.). Furthermore, the second liquid-liquid centrifugation step may be performed at a rate of from about 3,000 rpm to about 5,000 rpm (e.g., about 3,500 rpm to about 5,000 rpm, about 4,000 rpm to about 5,000 rpm, about 4,500 rpm to about 5,000 rpm, about 3,000 rpm to about 4,500 rpm, about 3,000 rpm to about 4,000 rpm, or about 3,000 rpm to about 3,500 rpm). For example, the demulsified supernatant may undergo a second liquid-liquid centrifugation step at a rate of about 4,100 rpm. The second liquid-liquid centrifugation step of the demulsified supernatant may be performed for about 5 minutes to about 30 minutes (e.g., about 5 minutes to about 25 minutes, about 5 minutes to about 20 minutes, about 5 minutes to about 15 minutes, about 5 minutes to about 10 minutes, about 10 minutes to about 30 minutes, about 15 minutes to about 30 minutes, about 20 minutes to about 30 minutes, or about 25 minutes to about 30 minutes). For example, the second liquid-liquid centrifugation may be performed for about 15 minutes.

### Demulsification

Additionally or alternatively, the squalene may be extracted from a squalene source of the disclosure, for example a fermentation composition, by demulsifying the supernatant of the fermentation composition that has been obtained by way of centrifugation. The demulsifying of the supernatant may be performed, for example, by contacting the supernatant obtained by way of centrifugation with a surfactant. The surfactant may be, for example, DOWFAX® 2A1, DOWFAX® 3B2, DOWFAX® 8390, DOWFAX® C6L, DOWFAX® C10L, TRITON® QS-15, TRITON® XN-45S, TERGITOL® L62, or any combination thereof. In particular embodiments, the surfactant is DOWFAX® 2A1.

DOWFAX® 2A1 includes a mixture of (i) water (about 52%), (ii) benzene, 1,1′-oxybis-, tetrapropylene derivatives, and sulfonate (about 46%, CAS #119345-04-9), (iii) sulfuric acid disodium salt (about 1%), and (iv) 2-methyl-4-isothiazolin-3-one (about 140 ppm, CAS #2682-20-4). DOWFAX® 3B2 includes a mixture of greater than or equal to 50% water, less than or equal to 38% benzenesulfonic acid, decyl(sulfophenoxy)-, disodium salt (CAS #36445-71-3), less than or equal to 8% benzenesulfonic acid, oxybis(decyl)-, disodium salt, and less than or equal to 1.5% sulfuric acid disodium salt. DOWFAX® 8390 includes greater than or equal to 59% water, between 15% and 35% disodium hexadecyldiphenyloxide disulfonate (CAS #65143-89-7), between 5% and 10% disodium dihexadecyldiphenyloxide disulfonate (CAS #70191-76-3), less than or equal to 1.5% sulfuric acid disodium salt, and less than 1% sodium chloride. DOWFAX® C6L includes less than or equal to 46% disodium hexyl diphenyl ether disulphonate (CAS #147732-60-3) and water. DOWFAX® C10L includes a mixture of greater than or equal to 50% water, less than or equal to 38% benzenesulfonic acid, decyl(sulfophenoxy), disodium salt, less than or equal to 8% benzene sulfonic acid, oxybis(decyl) disodium salt, and less than or equal to 1.5% sulfuric acid disodium salt. TRITON® QS-15 includes amines, C12-14-tert-alkyl, ethoxylated, sulfate, sodium salt (CAS #72379-24-9), less than or equal to 10% of amines, C12-14-tert-alkyl, ethoxylated (CAS #73138-27-9), less than or equal to 1% water, and less than or equal to 0.5% sulfuric acid. TRITON® XN-45S includes between 30% and 54% poly(oxy-1,2-ethanediyl), a-sulfo-w-(nonylphenoxy)-, branched, ammonium salt (CAS #68649-55-8), between 6% and 30% ethanol, 2-amino-, compd. with a-sulfo-w-(nonylphenoxy) poly(oxy-1,2-ethanediyl) (CAS #51617-74-4), and between 14% and 24% water. TERGITOL® L62 includes greater than or equal to 99% polyalkylene glycol (CAS #9003-11-6).

In some embodiments, the surfactant is added to the supernatant to a final concentration of from about 0.01% to about 10% (v/v) (e.g., about 0.01% to about 4% (v/v), about 0.01% to about 3% (v/v), about 0.01% to about 2% (v/v), about 0.01% to about 1% (v/v), about 0.01% to about 0.1% (v/V), about 0.1% to about 5% (v/v), about 1% to about 4% (v/v), about 1% to about 3% (v/v), or about 1% to about 2% (v/v)). For example, the surfactant may be added to the supernatant to a final concentration of between about 1% to about 2% (v/v) (e.g., about 1.1% (v/v), 1.2% (v/v), 1.3% (v/V), 1.4% (v/v), 1.5% (v/v), 1.6% (v/v), 1.7% (v/v), 1.8% (v/v), 1.9% (v/v), or 2% (v/v)).

The demulsifying of the supernatant may be performed at a pH of from about 6 to about 8 (e.g., a pH of about 6 to about 7.5, a pH of about 6 to about 7, a pH of about 6 to about 6.5, a pH of about 6.5 to about 8, a pH of about 7 to about 8, and a pH of about 7.5 to about 8). Furthermore, the demulsifying may be performed at a temperature of from about 50° C. to about 90° C. (e.g., about 50° C. to about 80° C., about 50° C. to about 70° C., about 50° C. to about 60° C., about 60° C. to about 90° C., about 70° C. to about 90° C., or about 80° C. to about 90° C.). In particular embodiments, the demulsifying is performed at a temperature of about 70° C.

### Base Wash

To further improve the extraction of squalene from a squalene source of the disclosure, a base wash may be utilized. The base wash may be performed by mixing an aqueous solution including a base with, for example, an oil component that is obtained from centrifugation of the squalene source, such as a fermentation composition. The base in the aqueous solution may contain a hydroxide salt, such as NaOH, LiOH, KOH, or Ca(OH)₂. In particular embodiments, the base is NaOH.

The concentration of hydroxide in the aqueous solution may be, for example, about 1 M. In some embodiments, the pH of the aqueous solution may be from about 9 to about 13 (e.g., about 9 to about 12, about 9 to about 11, about 9 to about 10, about 10 to about 13, about 11 to about 13, or about 12 to about 13). In some embodiments, the concentration of hydroxide (OH⁻) in the aqueous solution is between about 0.0001 M and 1 M (e.g., 0.0001 M and about 0.1 M, about 0.0001 M and about 0.01 M, about 0.0001 M and about 0.001 M, about 0.001 M and about 1 M, about 0.01 M and about 1 M, or about 0.1 M and about 1 M).

In some embodiments, the oil component obtained from liquid-liquid centrifugation is mixed with the aqueous solution containing a base at a ratio of from about 0.1:1 (v/v) to about 1:0.1 of the oil component to the aqueous solution, such as a ratio of about 0.1:1, 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, or 1:0.1 (v/v) of the oil component to the aqueous solution. In some embodiments, the oil component obtained from liquid-liquid centrifugation is mixed with the aqueous solution containing a base at a ratio of about 1:1 (v/v) of the oil component to the aqueous solution.

The oil component and the aqueous solution including a base may be mixed at a temperature of from about 20° C. to about 80° C. (e.g., about 20° C. to about 70° C., about 20° C. to about 60° C., about 20° C. to about 50° C., about 20° C. to about 40° C., about 20° C. to about 30° C., about 30° C. to about 80° C., about 40° C. to about 80° C., about 50° C. to about 80° C., about 60° C. to about 80° C., or about 70° C. to about 80° C.). For example, the oil component may be mixed with the aqueous solution including the base at a temperature of about 40° C.

The base wash may be formed by mixing the aqueous solution including a base with the oil component for about 0.25 hours or more. In some embodiments, the base wash may be for about 0.5 hours or more. In some embodiments, the oil component is mixed with the aqueous solution comprising the base for from about 0.1 hours to 10 hours (e.g., about 0.1 hours to about 9 hours, about 0.1 hours to about 8 hours, about 0.1 hours to about 7 hours, about 0.1 hours to about 6 hours, about 0.1 hours to about 5 hours, about 0.1 hours to about 4 hours, about 0.1 hours to about 3 hours, about 0.1 hours to about 2 hours, about 0.1 hours to about 1 hour, about 1 hour to about 10 hours, about 2 hours to about 10 hours, about 3 hours to about 10 hours, about 4 hours to about 10 hours, about 5 hours to about 10 hours, about 6 hours to about 10 hours, about 7 hours to about 10 hours, about 8 hours to about 10 hours, or about 9 hours to about 10 hours). For example, the aqueous solution including a base may be mixed with the oil component for about 1 hour.

### Evaporation

In some embodiments of the disclosure, evaporation is used to isolate squalene, such as following extraction of the squalene from a squalene source described herein. In some embodiments of the disclosure, the evaporation step includes distilling the squalene. In some embodiments of the disclosure, fractional distillation is used to isolate squalene. In some embodiments, simple distillation is used to isolate squalene. The distillation process may involve, for example, initially heating the squalene to a temperature of from about 20° C. to about 90° C. (e.g., about 20° C. to about 90° C., about 20° C. to about 70° C., about 20° C. to about 50° C., about 20° C. to about 30° C., about 30° C. to about 90° C., about 50° C. to about 90° C., or about 70° C. to about 90° C.). For example, the squalene may be initially heated to a temperature of from about 60° C. to about 70° C. (e.g., about 60° C., 61° C., 62° C., 63° C., 64° C., 65° C., 66° C., 67° C., 68° C., 69° C., or 70° C.).

In some embodiments, the squalene may be evaporated at a temperature of from about 150° C. to about 250° C. (e.g., about 150° C. to about 225° C., about 150° C. to about 200° C., about 150° C. to about 175° C., about 175° C. to about 250° C., about 200° C. to about 250° C., or about 225° C. to about 250° C.). For example, the squalene may be evaporated at a temperature of from about 200° C. to about 205° C. (e.g., about 200° C., 201° C., 202° C., 203° C., 204° C., or 205° C.). Evaporation of the squalene may be performed under vacuum. For example, in some instances, the evaporation is performed at a pressure of about 0.5 to about 5 torr (e.g., about 0.5 torr to about 1 torr, about 0.5 torr to about 2 torr, about 0.5 torr to about 3 torr, about 0.5 torr to about 4 torr, about 4 torr to about 5 torr, about 3 torr to about 5 torr, about 2 torr to about 5 torr, about 1 torr to about 5 torr, about 0.7 torr to about 4 torr, about 2 torr to about 4 torr, or about 0.7 torr to about 2 torr).

Following evaporation of the squalene, the squalene may be condensed and cooled. Condensation and cooling of the squalene may be performed at temperature of about 70° C. or less, such as a temperature of from about 20° C. to about 70° C. (e.g., about 20° C. to about 60° C., about 20° C. to about 50° C., about 20° C. to about 40° C., about 20° C. to about 30° C., about 30° C. to about 70° C., about 40° C. to about 70° C., about 50° C. to about 70° C., or about 60° C. to about 70° C.).

In some embodiments, the squalene is condensed and cooled at a temperature of from about 20° C. to about 25° C. (e.g., about 20° C., 21° C., 22° C., 23° C., 24° C., or 25° C.). Condensation and cooling of the squalene during evaporation may be performed under vacuum. For example, in some instances, the evaporation may be performed at a pressure of about 1 torr.

In some embodiments, evaporation of the squalene is performed using a wiped film evaporator.

### Chromatography

In some embodiments, chromatography is used to isolate squalene, such as following extraction and evaporation of the squalene from a squalene source of the disclosure. The chromatography step may include, for example, exposing the squalene to a resin (e.g., a polar resin) and recovering squalene from the resin. The resin may be aluminum oxide resin, such as basic aluminum oxide resin, an acidic aluminum oxide resin, or a neutral aluminum oxide resin. In some embodiments, the resin may be a silica resin. The particle size of the resin may be, for example, between about 50 μm to about 700 μm (e.g., about 50 μm to about 600 μm, about 50 μm to about 500 μm, about 50 μm to about 400 μm, about 50 μm to about 300 μm, about 50 μm to about 200 μm, about 50 μm to about 100 μm, about 100 μm to about 650 μm, about 200 μm to about 650 μm, about 300 μm to about 650 μm, about 400 μm to about 650 μm, about 500 μm to about 650 μm, or about 600 μm to about 650 μm). For example, the particle size of the resin may be between about 50 μm and about 200 μm (e.g., about 50 μm to about 150 μm, about 50 μm to about 100 μm, about 50 μm to about 75 μm, about 75 μm to about 200 μm, about 100 μm to about 200 μm, about 125 μm to about 200 μm, or about 150 μm to about 200 μm). The resin may require an activation step. The activation step may be, for example, drying of the resin prior to use.

In some embodiments, the chromatography is performed using a flow rate of from about 1 bed volumes per hour (BV/hr) to about 5 BV/hr (e.g., about 1 BV/hr to about 4 BV/hr, 1 BV/hr to about 3 BV/hr, about 1 BV/hr to about 2 BV/hr, about 2 BV/hr to about 5 BV/hr, about 3 to about 5 BV/hr, or about 4 BV/hr to about 5 BV/hr). In some embodiments, the chromatography is performed using a flow rate of from about 1.5 BV/hr to about 3 BV/hr (e.g., about 1.5 BV/hr to about 2.5 BV/hr, 1.5 BV/hr to about 2 BV/hr, about 2 BV/hr to about 3 BV/hr, or about 2.5 BV/hr to about 3 BV/hr). In some embodiments, the chromatography is performed using a flow rate of from about 2 BV/hr to about 2.5 BV/hr (e.g., about 2 BV/hr, 2.1 BV/hr, 2.2 BV/hr, 2.3 BV/hr, 2.4 BV/hr, or 2.5 BV/hr).

### Purity of Isolated Squalene

Disclosed herein are compositions that include squalene recovered from a squalene source, such as using the methods described above. In some embodiments, the compositions include (i) squalene recovered from a fermentation composition prepared using any of the provided methods, and (ii) one or more impurities. The concentration of the squalene relative to the total amount of the squalene and the one or more impurities in the composition may be, for example, from 90 wt % to 100 wt % or more, e.g., 90 wt %, 90.5 wt %, 91 wt %, 91.5 wt %, 92 wt %, 92.5 wt %, 93 wt %, 93.5 wt %, 94 wt %, 94.5 wt %, 95 wt %, 95.5 wt %, 96 wt %, 96.5 wt %, 97 wt %, 97.5 wt %, 98 wt %, 98.5 wt %, 99 wt %, 99.5 wt %, 99.9 wt %, or 100 wt %, or more. For example, the concentration of squalene relative to the total amount of the squalene and the one or more impurities in the composition may be from about 99.5 wt % to about 100 wt %, or more, for example, about 99.5 wt %, 99.6 wt %, 99.7 wt %, 99.8 wt %, 99.9 wt %, or 100 wt %.

In some embodiments, the squalene concentration relative to the total amount of the squalene and the one or more impurities can be up to 100 wt %, such as up to 100 wt %, 99 wt %, 98 wt %, 97 wt %, 96 wt %, 95 wt %, 94 wt %, 93 wt %, 92 wt %, 91 wt %, or 90 wt %. In some embodiments, the squalene concentration relative to the total amount of the squalene and the one or more impurities may be greater than 90 wt %, e.g., greater than 90 wt %, greater than 91 wt %, greater than 92 wt %, greater than 93 wt %, greater than 94 wt %, greater than 95 wt %, greater than 96 wt %, greater than 97 wt %, greater than 98 wt %, greater than 99 wt %, greater than 99.1 wt %, greater than 99.2 wt %, greater than 99.3 wt %, greater than 99.4 wt %, greater than 99.5 wt %, greater than 99.6 wt %, greater than 99.7 wt %, greater than 99.8 wt %, or greater than 99.9 wt %.

In some embodiments, a squalene-containing composition of the disclosure may contain squalene and one or more impurities, such that the concentration of the one or more impurities relative to the total amount of the squalene and the one or more impurities is from about 0.1 wt % to about 0.5 wt %, such as about 0.1 wt %, 0.2 wt %, 0.3 wt %, 0.4 wt %, or 0.5 wt %. In some embodiments, the concentration of the one or more impurities relative to the total amount of the squalene and the one or more impurities is less than 0.5 wt %, e.g., less than 0.4 wt %, less than 0.3 wt %, less than 0.2 wt %, or less than 0.1 wt %. In some embodiments, the concentration of the one or more impurities relative to the total amount of the squalene and the one or more impurities is about 0.1 wt %, about 0.2 wt %, about 0.3 wt %, about 0.4 wt %, or about 0.5 wt %.

### Vaccine Preparation

The squalene produced by any one of the methods described herein may be used as an adjuvant. The squalene of the disclosure may be admixed with an immunogenic antigen (e.g., a protein derived from a pathogen or cancer cell, or a nucleic acid encoding the same) so as to produce a vaccine for therapeutic or prophylactic use. In some embodiments, the squalene is covalently conjugated to an antigen, thereby forming a self-adjuvanting vaccine. Self-adjuvanting vaccines may exhibit the advantageous effect of being rapidly internalized by antigen-presenting cells of the immune system, such as macrophages and dendritic cells, among others. Moreover, the use of self-adjuvanting vaccines helps to ensure that the antigen-presenting cells activated by the adjuvant are the same cells that are exposed to antigen, thereby promoting an immune response that is highly specific for a desired antigen.

### Self-Adjuvanting Vaccine Synthesis

Self-adjuvanting vaccines of the disclosure may be produced, for example, by reacting an adjuvant (e.g., squalene) of the disclosure with a desired antigen, such as a protein expressed by a virus, bacterium, or protozoan (or a nucleic acid (e.g., a DNA or RNA molecule) encoding the same) or a protein expressed by a cancer cell (or a nucleic acid (e.g., a DNA or RNA molecule) encoding the same). The adjuvant may contain, for example, a reactive chemical substituent, such as a nucleophilic substituent, an electrophilic substituent, or dienophilic substituent, among others. The antigen may contain, for example, a chemical substituent that is suitable for reaction with the reactive substituent on the adjuvant, such that reacting the adjuvant and the antigen results in the formation of a stable covalent bond.

### Viral, Bacterial, and Protozoan Antigens

Exemplary antigens that may be used in conjunction with the vaccines of the disclosure include, without limitation, proteins that are expressed by a virus, bacterium, or protozoan, as well as nucleic acids (e.g., DNA or RNA molecules) encoding the same.

For example, in some embodiments, the antigen is a protein expressed by a virus selected from influenza virus, hepatitis A virus, hepatitis B virus, hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabia virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus, rhinovirus, mumps virus, poliovirus, human enterovirus, coxsackievirus, human papilloma virus, adeno-associated virus, astrovirus, JC virus, BK virus, SV40 virus, Norwalk virus, rotavirus, human immunodeficiency virus (HIV), and human T-lymphotropic virus. In some embodiments, the antigen is encoded by a nucleic acid (e.g., a DNA or RNA molecule) encoding the same.

In some embodiments, the antigen is a protein expressed by a coronavirus, such as SARS-CoV-2, MERS-COV, SARS-COV, OC43, or HKU1. In some embodiments, the antigen is encoded by a nucleic acid (e.g., a DNA or RNA molecule) encoding the same.

In some embodiments, the antigen is a protein expressed by a bacterium belonging to a genus selected from Mycobacterium (e.g., Mycobacterium tuberculosis), Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus. In some embodiments, the antigen is encoded by a nucleic acid (e.g., a DNA or RNA molecule) encoding the same.

In some embodiments, the antigen is a protein expressed by a protozoan. The antigen may be a protein expressed by a parasite, such as a parasite selected from the group consisting of Plasmodium malariae, Plasmodium vivax, Plasmodium ovale, Plasmodium falciparum, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Trichomonas vaginalis, and Histomonas meleagridis. The parasite may be a helminthic parasite, such as Richuris trichiura, Ascaris lumbricoides, Enterobius vermicularis, Ancylostoma duodenale, Necator americanus, Strongyloides stercoralis, Wuchereria bancrofti, Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, or Echinococcus granulosus. In some embodiments, the antigen is encoded by a nucleic acid (e.g., a DNA or RNA molecule) encoding a protein expressed by any of the above parasites.

### Cancer Vaccines

The present disclosure also features vaccines useful for the treatment and prevention of cancer. Such vaccines may include, for example, squalene admixed with, or conjugated to, a cancer antigen. Exemplary cancer antigens useful in conjunction with the compositions and methods of the disclosure include proteins expressed by a cancer cell, as well as nucleic acids (e.g., DNA or RNA molecules) encoding the same. Exemplary cancer cell antigens that may be used in the formation of a vaccine (e.g., a self-adjuvanting vaccine) described herein include, without limitation, gp100, Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-1, MUC-16, L1CAM, HERV-K-MEL, KK-LC-1, KM-HN-1, LAGE-1, Sp17, TAG-1, TAG-2, ENAH (hMena), mammaglobin-A, NY-BR-1, BAGE-1, MAGE-A1, MAGE-A2, MAGE-A4, MAGE-A6, MAGE-A9, MAGE-A10, MAGE-A12, MAGE-C2, mucink, SSX-2, SSX-4, TRAG-3, c-myc, cyclin B1, p62, Survivin, CD45, DKK1, RU2AS, Telomerase, K-ras, G250, Hepsin, Intestinal carboxyl esterase, α-foetoprotein, M-CSF, PSMA, CASP-5, COA-1, OGT, OS-9, TGF-βRII, gp70, CALCA, CD274, mdm-2, α-actinin-4, Elongation factor 2, ME1, NFYC, GAGE-1/2/8, GAGE-3/4/5/6/7, XAGE-1b/GAGED2a, STEAP1, PAP, PSA, FGF5, hsp70-2, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, GnTVf, LY6K, and NA88-A. In some embodiments, the antigen is encoded by a nucleic acid molecule (e.g., a DNA or RNA molecule) encoding any of the above proteins.

Additional examples of tumor-specific antigens are described in Wilkinson et al. Cancer Immunol. Immunother. 61(2):169-79 (2012); Hural et al. J. Immunol. 169(1):557-65 (2002); Tsukahara et al. Cancer Res. 64(15):5442-8 (2004); Kessler et al. J. Exp. Med. 193(1):73-88 (2001); Ikeda et al. Immunity 6(2):199-208 (1997); Asemissen et al. Clin. Cancer Res. 12(24):7476-82 (2006); Ohminami et al. Blood. 95(1):286-93 (2000); Guo et al. Blood. 106(4):1415-8 (2005); Lin et al. J. Immunother. 36(3):159-70 (2013); Fujiki et al. J. Immunother. 30(3):282-93 (2007); Wick et al. Clin. Cancer Res. 20(5):1125-34 (2014); Hassan et al. Appl. Immunohistochem. Mol. Morphol. 13(3):243-7 (2005); Jager et al. J Exp Med. 187(2):265-70 (1998); Jager et al. Proc. Natl. Acad. Sci. U.S.A. 103(39):14453-8 (2006); Chen et al. J Immunol. 165(2):948-55 (2000); and Mandic et al. J Immunol. 174(3):1751-9 (2005), each of which is incorporated herein by reference as it pertains to tumor-specific antigens.

In some embodiments, the cancer antigen is a protein expressed by an ovarian cancer cell. Such proteins include Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-16, L1CAM, Mannan-MUC-1, HERV-K-MEL, KK-LC-1, KM-HN-1, LAGE-1, MAGE-A4, SSX-4, TAG-1, and TAG-2, among others. In some embodiments, the antigen is encoded by a nucleic acid molecule (e.g., a DNA or RNA molecule) encoding any of the foregoing proteins.

In some embodiments, the cancer antigen is a protein expressed by a breast cancer cell. Such proteins include ENAH (hMena), mammaglobin-A, NY-BR-1, EpCAM, NY-ESO-1, BAGE-1, HERV-K-MEL, KK-LC-1, KM-HN-1, LAGE-1, MAGE-A1, MAGE-A2, mucink, Sp17, SSX-2, TAG-1, TAG-2, TRAG-3, Her2/Neu, c-myc, cyclin B1, MUC1, p53, p62, and Survivin, among others. In some embodiments, the antigen is a nucleic acid molecule (e.g., a DNA or RNA molecule) encoding any of the foregoing proteins.

In some embodiments, the cancer antigen is a protein expressed by a pancreatic cancer cell. Such proteins include ENAH (hMena), PBF, K-ras, Mesothelin, and mucink, among others. In some embodiments, the antigen is a nucleic acid molecule (e.g., a DNA or RNA molecule) encoding any of the foregoing proteins.

In some embodiments, the cancer antigen is a protein expressed by a colorectal cancer cell. Such proteins include ENAH (hMena), Intestinal carboxyl esterase, CASP-5, COA-1, OGT, OS-9, TGF-βRII, NY-ESO-1, CEA, HERV-K-MEL, KK-LC-1, KM-HN-1, LAGE-1, MAGE-A2, Sp17, TAG-1, TAG-2, c-myc, cyclin B1, MUC1, p53, p62, Survivin, and gp70, among others. In some embodiments, the antigen is encoded by a nucleic acid molecule (e.g., a DNA or RNA molecule) encoding any of the foregoing proteins.

In some embodiments, the cancer antigen is a protein expressed by a lung cancer cell. Such proteins include CD274, mdm-2, a-actinin-4, Elongation factor 2, ME1, NFYC, NY-ESO-1, GAGE-1/2/8, HERV-K-MEL, KK-LC-1, KM-HN-1, LAGE-1, MAGE-A2, MAGE-A6, Sp17, TAG-1, TAG-2, TRAG-3, XAGE-1b/GAGED2a, c-myc, cyclin B1, Her2/Neu, MUC1, p53, p62, and Survivin, among others. In some embodiments, the antigen is encoded by a nucleic acid molecule (e.g., a DNA or RNA molecule) encoding any of the foregoing proteins.

In some embodiments, the cancer antigen is a protein expressed by a prostate cancer cell. Such proteins include DKK1, ENAH (hMena), Kallikrein 4, PSMA, STEAP1, PAP, PSA, NY-ESO-1, BAGE-1, GAGE-1/2/8, GAGE-3/4/5/6/7, HERV-K-MEL, KK-LC-1, KM-HN-1, LAGE-1, and Sp17, among others. In some embodiments, the antigen is encoded by a nucleic acid molecule (e.g., a DNA or RNA molecule) encoding any of the foregoing proteins.

In some embodiments, the cancer antigen is a protein expressed by a melanoma cell. Such proteins include gp100, Hepsin, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, NY-ESO-1, BAGE-1, GAGE-1/2/8, GAGE-3/4/5/6/7, GnTVf, HERV-K-MEL, KK-LC-1, KM-HN-1, LAGE-1, LY6K, MAGE-A1, MAGE-A6, MAGE-A10, MAGE-A12, MAGE-C2, NA88-A, Sp17, SSX-2, SSX-4, and TRAG-3, among others. In some embodiments, the antigen is encoded by a nucleic acid molecule (e.g., a DNA or RNA molecule) encoding any of the foregoing proteins.

In some embodiments, the cancer antigen is a protein expressed by a squamous cell carcinoma cell. Such proteins include CASP-8, p53, and SAGE, among others. In some embodiments, the antigen is encoded by a nucleic acid molecule (e.g., a DNA or RNA molecule) encoding any of the foregoing proteins.

In some embodiments, the cancer antigen is a protein expressed by a chronic myeloid leukemia cell. Such proteins include BCR-ABL, dek-can, EFTUD2, and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by an acute lymphoblastic leukemia cell. Such proteins include ETV6-AML1, and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by an acute myelogenous leukemia cell. Such proteins include FLT3-ITD, Cyclin-A1, and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by a chronic lymphocytic leukemia cell. Such proteins include FNDC3B and GAGE-3/4/5/6/7, among others. In some embodiments, the antigen is encoded by a nucleic acid molecule (e.g., a DNA or RNA molecule) encoding any of the foregoing proteins.

In some embodiments, the cancer antigen is a protein expressed by a multiple myeloma cell. Such proteins include MAGE-C1, NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments, the cancer antigen is a protein expressed by a bladder cancer cell. Such proteins include BAGE-1, GAGE-1/2/8, GAGE-3/4/5/6/7, MAGE-A4, MAGE-A6, SAGE, NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

In some embodiments, the cancer antigen is a protein expressed by a neuroblastoma cell. Such proteins include NY-ESO-1, LAGE-1, HERV-K-MEL, KK-LC-1, KM-HN-1, and Sp17, among others. In some embodiments, the antigen is encoded by a nucleic acid molecule (e.g., a DNA or RNA molecule) encoding any of the foregoing proteins.

Additionally or alternatively, a cancer vaccine of the disclosure may contain an adjuvant compound admixed with, or conjugated to, an agent that activates antigen-presenting cells of the immune system, such as a toll-like receptor 4 (TLR4) agonist. The immune-stimulating agent may be present in addition to, or instead of, the cancer antigen. Exemplary TLR4 agonists useful in conjunction with the compositions and methods of the disclosure include glucopyranosyl lipid A and lipopolysaccharide, among others.

### Protein and Nucleic Acid Antigens

Antigens that may be used in the formation of a vaccine of the disclosure include proteins (e.g., a protein expressed by a virus, bacterium, protozoan, or cancer cell described herein). In some embodiments, the antigen is encoded by a nucleic acid encoding such a protein. The nucleic acid may be, for example, a DNA molecule or RNA molecule encoding a protein expressed by a virus, bacterium, protozoan, or cancer cell, such as any of the proteins recited above. Exemplary methods for producing RNA vaccines are described, for example, in Erasmus et al. Molecular Therapy 26:1-16 (2018), the disclosure of which is incorporated herein by reference as it pertains to nucleic acid vaccines.

Nucleic acids encoding a protein of interest (e.g., a protein expressed by a virus, bacterium, protozoan, or cancer cell described herein) may be produced using synthetic chemistry and/or molecular biology techniques known in the art. For example, once a desired protein is identified, an open reading frame (ORF) encoding the protein may be designed using standard codon-amino acid relationships known in the art. The ORF may be a wild-type ORF that occurs naturally for the selected protein, an isoform, or a variant or fragment thereof.

In some embodiments, the nucleotide sequence of the ORF is codon optimized for expression in a desired cell type (e.g., a mammalian cell, such as a human cell). Codon optimization methods are known in the art. Codon optimization may be used, for example, to match codon frequencies in target and host organisms to ensure proper protein folding, bias GC content to increase RNA stability or reduce secondary structures, minimize tandem repeat codons or base runs that may impair gene construction or expression, customize transcriptional and translational control regions, insert or remove protein trafficking sequences, remove or add post-translation modification sites in encoded proteins (e.g. glycosylation sites), add, remove or shuffle protein domains, insert or delete restriction sites, modify ribosomal binding sites and RNA degradation sites, and/or adjust translational rates to allow the various domains of the protein to fold properly.

### Lipid Nanoparticle Formulations

In some embodiments of the disclosure, nucleic acid (e.g., DNA or RNA) vaccines are formulated in a nanoparticle, such as a lipid nanoparticle. The nanoparticle (e.g., lipid nanoparticle) may be constructed such that the squalene of the disclosure is located within the core of the nanoparticle, and the nucleic acid (e.g., DNA or RNA) component is located on the nanoparticle's interior or exterior.

In some embodiments, the nanoparticle (e.g., lipid nanoparticle) includes a polycation. The nanoparticle may be, for example, a lipid-polycation complex. The lipid nanoparticle may be manufactured, for example, using methods described in US 2012/0178702, the disclosure of which is incorporated herein by reference as it pertains to nanoparticle formulations and techniques for producing the same. As a non-limiting example, the polycation may include a cationic peptide or a polypeptide such as polylysine, polyornithine, or polyarginine. In some embodiments, the cationic peptide is one described in WO 2012/013326 or US 2013/0142818, each of which is incorporated herein by reference as it pertains to cationic peptides. In some embodiments, nanoparticle formulations of the disclosure include a non-cationic lipid, such as cholesterol or dioleoyl phosphatidylethanolamine, among others.

### Routes of Administration

Vaccines produced using squalene as an adjuvant of the disclosure may be administered to a subject (e.g., a mammalian subject, such as a human) for therapeutic or prophylactic treatment. Such vaccines may be administered to a subject by way of any suitable route of administration. Exemplary routes of administration useful in conjunction with the vaccines of the disclosure include, without limitation, injection by way of the intramuscular, intraperitoneal, intradermal, or subcutaneous routes, or by way of transmucosal administration to the oral, respiratory, or genitourinary tract(s).

### Additional Excipients

Vaccine compositions of the present disclosure may contain, in addition to an adjuvant compound describe herein, one or more pharmaceutically acceptable carriers, diluents, excipients, or solvents. Exemplary pharmaceutically acceptable carriers, diluents, excipients, and solvents that may be used in conjunction with the vaccines of the present disclosure include those pharmaceutically acceptable additives described in Adejare, Aldeboye, Remington: The Science and Practice of Pharmacy (Academic Press, 2020).

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Purification of Squalene from Fermentation Broth

In this example, squalene was produced by fermentation of yeast cells, and was subsequently isolated by a multi-step process. The process included extraction of the squalene from the fermentation broth, followed by distillation of the extracted squalene, and chromatographic purification of the distilled squalene. As is outlined in further detail below, the extraction step included homogenization of the fermentation broth, followed by centrifugation, and caustic washing. Distillation and chromatographic purification of the squalene resulted in a final product purity of >99.5% by weight. This process is the first time high-purity (>99.5% by weight) squalene has been produced from a renewable source (in this instance, sugar) by way of fermentation.

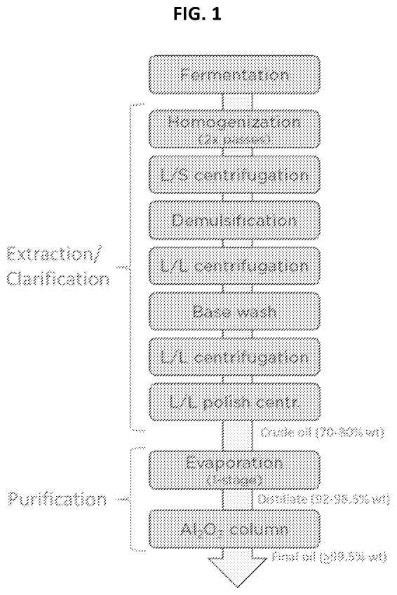

An overview of the multi-step purification process described in this example is shown in FIG. 1. Each of the steps in this process is described in further detail in the sections that follow.

### Homogenization

In this example, yeast cells were fermented in the presence of sugar so as to biosynthesize squalene. Whole-cell broth from the fermentation was composed of 35-40% by volume solids (biomass), with a squalene content in the broth of 40-50 g/kg squalene. About 50-80% of the squalene was localized in the solid biomass, and the remaining squalene was present as an emulsion in the broth.

To maximize squalene recovery, the first step of the purification process involved a two-pass, high-pressure homogenization. The homogenization step was designed to shear the yeast cells and release all of the squalene into the liquid portion of the broth. Experiments were performed with a GEA PandaPLUS 2000 homogenizer (1-2 L scale) to optimize the homogenization conditions. For example, homogenization was tested at various pH levels from pH 3 to pH 9. In addition, homogenization was tested at various temperatures from 5° C. to 70° C. The results of select experiments are shown in Table 1, below.

TABLE 1
Percentage of squalene released to supernatant as a function
of homogenization conditions and number of passes

| Homogenization conditions | 1-pass | 2-pass | 3-pass |
|---|---|---|---|
| 400 bar, ambient T, 35% by volume | | | 71% |
| solids feed, native pH | | | |
| 900 bar, ambient T, 35% by volume | 71% | 83% | 93% |
| solids feed, native pH | | | |

These findings were then validated with the GEA Ariete NS3006L (Panther) homogenizer (100-1000 L scale). The optimized homogenization conditions involved (i) diluting the whole-cell broth with H₂O to 30-35% solids (by volume) and (ii) homogenizing the broth in 2 steps, each conducted at 900 bar at ambient temperature. This process resulted in a homogenized fermentation broth in which 80-85% of the fermented squalene could be recovered from the liquid component of the broth.

The centrifugation process for separating the liquid component from the solid (biomass) component is described in further detail below.

### Liquid-Solid Centrifugation

Following homogenization, the whole-cell broth (homogenate) was centrifuged to remove sedimentable solids. This resulted in recovery of an aqueous-emulsion liquid phase, about 30-50% by volume emulsion. Prior to centrifugation, the homogenate was batch-heated to temperatures ranging from 18-75° C. to improve liquid-solid separation, as well as to promote portioning of the squalene into the liquid component.

Lab scale volumes (1-2 L) were prepared by centrifugation for 15 min at 4100 rpm, which was sufficient to clearly separate the emulsion, aqueous, and cell pellet portions.

At pilot scale, a continuous centrifuge was used (Alfa Laval DX-203 disc stack centrifuge with 3×0.6 mm nozzles). Prior to centrifugation at pilot scale, the homogenate feed was diluted with H₂O to 25% by volume solids before heating. After centrifugation, the solids-rich stream was discarded as waste, and the emulsion-rich light phase (<1% by volume solids) was carried on to demulsification.

### Demulsification

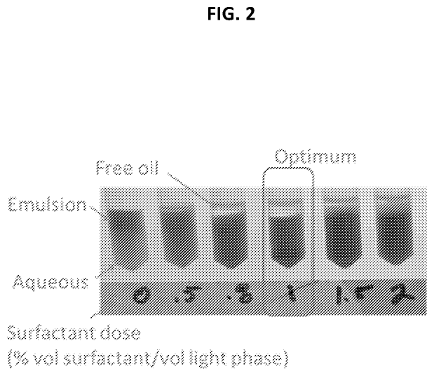

Following solid-liquid centrifugation, the emulsion of the light phase was broken to liberate the squalene oil. To achieve this, a surfactant and heating were used. The light-phase was screened at 2-mL scale for the appropriate surfactant and surfactant dosage to break the emulsion, as shown in FIG. 2.

Table 2 lists the conditions and results of several surfactants that were tested at concentrations of from 0.1% to 3% by volume. The surfactants that were tested included DOWFAX® 2A1, DOWFAX® 3B2, DOWFAX® 8390, DOWFAX® C6L, DOWFAX® C10L, TRITON® QS-15, TRITON® XN-45S, and TERGITOL® L62. The surfactants were tested at ambient and elevated temperatures (50° C., 70° C.), as well as at a variety of pH's (3, native, 10, 12, and 13). The optimal condition to break the emulsion was found to include 1-2% by volume DOWFAX® 2A1 at native pH and 70° C., which is shown in line 1 of Table 2, below.

TABLE 2
Conditions and results of surfactant screening

| | Temp | Conc | |
|---|---|---|---|
| Surfactant | (° C.) | (% v/v) | pH |
| DOWFAX ® 2A1 | 70 | 0-2.0 | native |
| DOWFAX ® 2A1 | 70 | 0-2.0 | 12.3 |
| DOWFAX ® 2A1 | 70 | 0-2.0 | 13 |
| DOWFAX ® C6L | 70 | 0-2.0 | native |
| DOWFAX ® 3B2 | 70 | 0-2.0 | native |
| DOWFAX ® 3B2 | 70 | 0-2.0 | 12.3 |
| DOWFAX ® C10L | 70 | 0-2.0 | native |
| DOWFAX ® C10L | 70 | 0-2.0 | 12.3 |
| DOWFAX ® C10L | 70 | 0-2.0 | 13 |
| DOWFAX ® 8390 | 70 | 0-2.0 | native |
| DOWFAX ® 8390 | 70 | 0-2.0 | 12.3 |
| TRITON ® QS-15 | 20 | 0-1.0 | 10 |
| TRITON ® QS-15 | 70 | 0-1.0 | 10 |
| TRITON ® QS-15 | 70 | 0-2.0 | 12.3 |
| TRITON ® QS-15 | 70 | 0-2.0 | 13 |
| TRITON ® XN-45S | 70 | 0-2.0 | 12.3 |
| TRITON ® X-114 | 70 | 0-2.0 | 13 |

### Liquid-Liquid Centrifugation

After the light-phase was mixed with the surfactant under the conditions described above, the squalene stream was centrifuged, separating the material into a crude oil stream and an aqueous stream. This centrifugation step was performed at an elevated temperature (70° C.) directly following the demulsification step above.

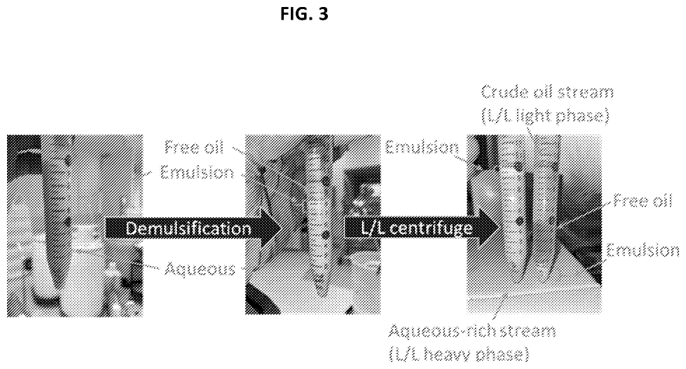

A bench-top centrifuge was used at 4100 rpm for 15 min. At the pilot scale, a continuous centrifuge (Alfa Laval LAPX liquid-liquid centrifuge with a 61 mm gravity ring) was used. The crude oil generated at this step was typically 75-80% by weight squalene, with minimal emulsion carry-over into the crude oil (FIG. 3). The aqueous stream, which was discarded as waste, was 5-10% by volume emulsion and <1% by volume crude oil. A polishing centrifuge (Alfa Laval Clara 20) was used to remove aqueous/emulsion carry over into the crude oil when there was >5% vol aqueous emulsion in the L/L crude oil from the LAPX.

### Base Wash

The crude oil generated from the liquid-liquid centrifugation step above was yellow in color and contained 6-10% by weight fatty acids. Mixing the crude oil with 1:1 vol/vol base solution (1 M NaOH, pH 13) at 20-70° C. for 0.5-2 hour resulted in a colorless crude oil with minimal emulsion (Table 3). Other ratios of crude oil to base, ranging from 0.5:1 to 1:0.5, were tested. In some of the experiments, during the base wash, the crude oil was mixed with the base at a ratio of about 1:0.5 vol/vol crude oil to 1 M NaOH, mixing vigorously at 40° C. for at least 1 hour. NaOH solutions having a pH of 9 and a pH of 12, respectively, were insufficient to improve squalene titer-rather, these solutions resulted in negligible color change and a thick emulsion, respectively. Those of skill in the art will appreciate that depending on the base, pH, ratio of crude oil to base, and temperature, the required hold time for the base wash may be less than 0.5 hours or more than 2.0 hours.

The results of these base-wash experiments are shown in Table 3, below.

TABLE 3
Conditions and results of base wash studies

| | Ratio | | Hold | Squalene | |
|---|---|---|---|---|---|
| | (crude | Temp | Time | titer | |
| Base, [M] | oil:base) | (° C.) | (h) | (g/kg) | Observation |
| NaOH, 1.0M | 1:1 | 25 | 0.5 | 779.9 | Colorless oil, slight turbidity |
| NaOH, 1.0M | 1:1 | 25 | 2 | 786.3 | Colorless oil, slight turbidity |
| NaOH, 1.0M | 1:1 | 70 | 0.5 | 790.5 | Colorless oil, slight turbidity |
| NaOH, 1.0M | 1:1 | 70 | 2 | 792.2 | Colorless oil, slight turbidity |
| NaOH, 0.1M | 1:0.5 | 40 | 1 | 576.0 | Stable white emulsion, no free |
| | | | | | oil |
| NaOH, 0.1M | 1:0.5 | 40 | 2 | 657.6 | Stable white emulsion, no free |
| | | | | | oil |
| NaOH, 0.01M | 1:0.5 | 70 | 1 | 762.0 | Stable white emulsion, no free |
| | | | | | oil |
| NaOH, | 1:0.5 | 25 | 2 | 780.6 | Yellow oil |
| 0.0001M | | | | | |
| NaOH, | 1:0.5 | 40 | 1 | 777.3 | Yellow oil |
| 0.0001M | | | | | |
| NaOH, | 1:0.5 | 70 | 0.5 | 774.7 | Yellow oil |
| 0.0001M | | | | | |
| NaOH, | 1:0.5 | 70 | 2 | 781.6 | Yellow oil |
| 0.0001M | | | | | |

### Liquid-Liquid Centrifugation

After the crude oil was mixed with the base solution under the conditions described above, the squalene stream was centrifuged, thereby separating the material into a base-washed crude oil stream and an aqueous stream. This centrifugation step was performed at temperatures ranging from 25° C. to 70° C., batch-heated from the base wash step described above. A bench-top centrifuge was used at 4100 rpm for 15 min. At the pilot scale a continuous centrifuge (Alfa Laval LAPX liquid-liquid centrifuge with a 60-61 mm gravity ring) was used.

The crude oil generated at this step was typically 75-85% by weight squalene with minimal emulsion carry-over into the crude oil. The highest percent by weight squalene was achieved when centrifugation was performed at 40° C. to 50° C. The aqueous stream, which was discarded as waste, was 1% by volume emulsion and <1% by volume crude oil. A polishing centrifuge (Alfa Laval Clara 20) was used to remove aqueous/emulsion carry over into the crude oil when there was >1% vol aqueous emulsion in the L/L crude oil from the LAPX.

### Squalene Evaporation

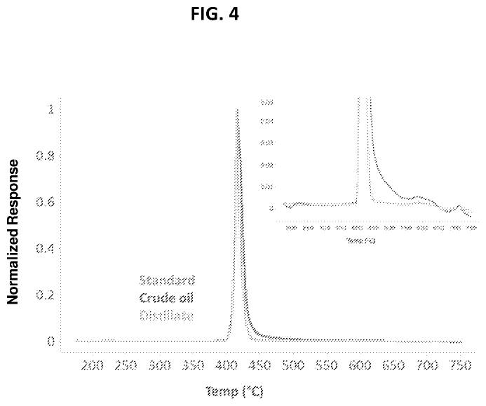

A key step in the purification process was the evaporation of the squalene from the crude oil resulting from the extraction steps described above. Pure squalene has a boiling point of 421° C. at 1 atm; the effect of temperature on boiling point using theoretical estimates demonstrates that reducing the pressure to 1 torr would reduce the boiling point to 200° C. (FIG. 4).

Development of this evaporation step was carried out using a 2″ glass WFE that has an electric band heater, and 77-82% wt crude squalene oil as generated from upstream fermentation/extraction (Table 4). Table 4 shows the typical quantity of distillate collected from the 2″ WFE process.

TABLE 4
Characterization of crude oil (WFE feed), distillate
(product stream), and heavies (waste)

| Parameter | Crude oil | Distillate* | Heavies* |
|---|---|---|---|
| Squalene purity (% wt) | 77-82 | 96-98 | 51-67 |
| H₂O content (% wt H₂O) | 0.016 | — | — |
| | (n = 1, polished crude) | | |
| Viscosity at 20° C. (cP) | 23.8 | 14.7 | 62.6 |
| Viscosity at 70° C. (cP) | 5.8 | 4.2 | 11.0 |
| Density at 20° C. (g/mL) | 0.872 | 0.857 | 0.903 |
| Density at 70° C. (g/mL) | 0.837 | 0.822 | 0.868 |
| Solids/emulsion (% vol) | 0 | 0 | 0 |

*Distillate and Heavies are generated from the 2″ WFE using the parameters outlined in Table 2

Table 5 summarizes the evaporation process as used on the 2″ Wiped Film Evaporator System (WFE). The base-washed crude was batch-heated in a H₂O bath to 60-70° C. and was held at this temperature while feeding the evaporator. The evaporator was heated to an average temperature of 200° C., which was sufficient to evaporate squalene at 1 torr. Those skilled in the art will appreciate that higher temperatures will achieve evaporation at higher pressures (e.g., 2-4 torr or more), while lower temperatures will achieve evaporation at lower pressures (e.g., 0.1-0.5 torr or less). The evaporator had a temperature range across the body of 150-250° C. The distillate stream had to be cooled to ≤70° C. (preferably to 20-25° C.) while under vacuum (for example, under N₂), as exposure to O₂ while at elevated temperature could lead to product degradation.

TABLE 5
2″ glass wiped-film evaporator (WFE)
operation and process parameters for evaporation

| | Target or | |
|---|---|---|
| Parameter | Range | Unit |
| Feed rate | 10-15 | mL/min |
| Feed Temperature (initial | 60-70 | ° C. |
| heating optional) | | |
| Feed density at 70° C. | 0.83-0.84 | g/mL |
| Feed viscosity at 70° C. | 5.7-7.1 | cP |
| Body Temperature target | 200-205 | ° C. |
| Body Temperature range | +/−20-50 | ° C. |
| Internal condenser temperature | 20-70 | ° C. |
| Wiper speed | 60-70 | % |
| Pressure | 0.95-1.05 | torr |
| Distillate:Feed | 0.50-0.60 | w/W |

Development of the evaporation procedure included the addition of a lights cut at 130° C. during evaporation to remove any potential impurities with lower boiling points than squalene in the feed. The lights cut was attempted as an initial evaporation step prior to squalene distillation at 200° C. The lights cut did not result in a change in the relative area percentage of squalene (˜97%), while a decrease in purity of from 95% by weight to 89% by weight was observed. Similarly, no change in the relative area percentage of squalene or purity was observed when attempting the lights cut after squalene distillation.

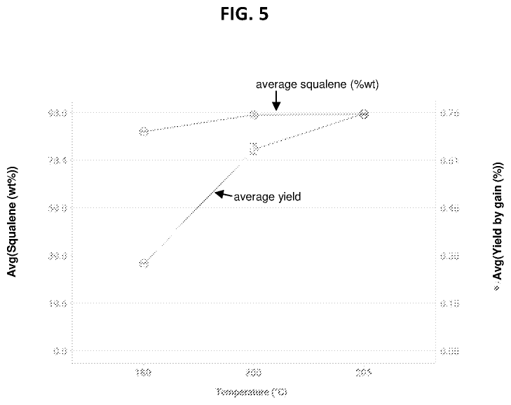

FIG. 5 shows results that were achieved using a single-stage squalene distillation performed at 180° C., 200° C., and 205° C. Distillate purity increased from 180° C. to 205° at ˜90% to 96%-97% by weight squalene, respectively. Yields improved as evaporation temperature was increased, with yields below 30% at 180° C. and up to 76% at 205° C. A 10% increase in yield was observed between 200° C. and 205° C. with little change in purity, indicating a potential to improve yield by increasing the operating temperature.

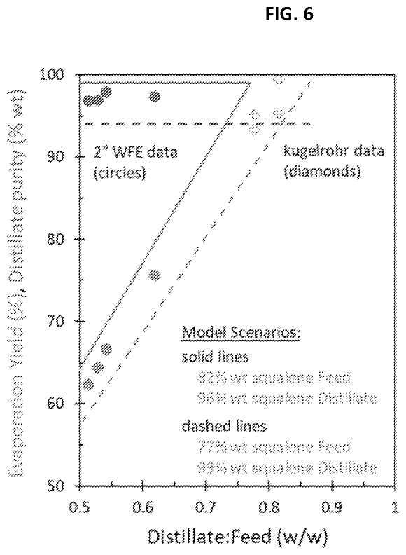

FIG. 6 shows the 2″ WFE data and data collected using the Kugelrohr, a bench-scale unit that subjects samples to deep vacuum (<1 torr) and high temp (air kettle heated). The resulting evaporation yield and distillate purity was plotted against the distillate to feed ratio. The distillate to feed ratio can be used as an in-process indicator of evaporation yield, but the actual yield is only determined with the feed and distillate concentrations. FIG. 6 also shows model estimates defining the expected ranges of the feed (crude oil) and distillate purities, generating an expectation envelope. The Kugelrohr data offers an estimate of what might be possible with a better temperature-controlled evaporation system, where the yield was increased substantially (70-80% target to >90%) with negligible impact on distillate purity.

### Alumina Column Polish

To this point, the evaporated squalene was 94-99% by weight pure, with the impurities being oxygenated species (e.g., fatty acids, sterols). The squalene was further purified using a fixed bed of basic aluminum oxide (alumina), which also deodorizes the squalene oil. The column was operated nearly isothermally at ambient temperature in down flow. The alumina resin used in development was an Alumina Basic Act I, which had a particle size of 50-200 μm (Table 6).

TABLE 6
Alumina resin specifications and performance results using
non-base washed oil feed and base washed oil feeds

| | | | | Column performance | | | | | |
|---|---|---|---|---|---|---|---|---|---|
| | | | | Non base washed oil feed | | | Base washed oil feed | | |
| | | H₂O | Size | | Yield | Final oil | | Yield | Final oil |
| Type | Activity | (% wt) | (um) | BV | (%) | purity (%) | BV | (%) | purity (%) |
| Basic | Super | ≤1 | 50-200 | 5 | 83 | 99.3 | 12 | 92 | |
| | Act. I | | | | | | | | |
| Basic | Act. I | 1-3 | 50-200 | 5 | 83 | | 12 | 92 | 100.0 |
| Basic | Act. III | 6-8 | 150-325 | 3 | 75 | | — | — | — |
| Basic | Act. III | 6-8 | 325-700 | 3 | 75 | | 12 | 91 | 99.8 |
| Basic/ESM | Act. III | 6-8 | 300-700 | — | — | | 6 | 90 | 99.9 |
| Neutral | Super | ≤1 | 50-200 | 3 | 75 | | — | — | — |
| | Act. I | | | | | | | | |
| Acidic | Act. I | 1-3 | 50-200 | 1 | 50 | | — | — | — |

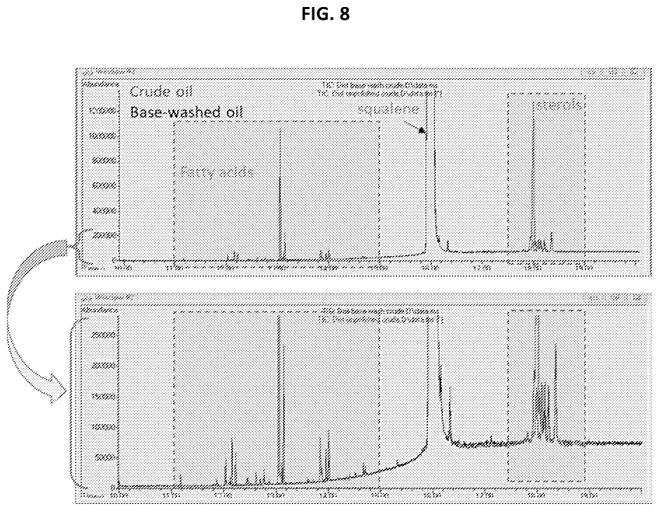

Note:
BV (bed-volume) is the number of bed volumes of feed passed through the alumina column before impurities were detected in the final squalene oil by GC-MS (FIG. 8)

The adsorption process was operated at a range of temperatures (15° C. to 25° C.), without active heating or cooling. The wetting step was mildly exothermic with a maximum temperature rise of ˜4° C. observed in the laboratory scale column, based on the skin temperatures recorded on a 2.5×29 cm glass column. This initial exotherm subsided once the first bed volume of feed had been processed and the column had been fully wetted. The feed should be processed more slowly during the first bed volume (pre-wetting), when the column is not yet filled with liquid.

The chromatography process consisted of an impurity adsorption step through a basic alumina bed followed by bed draining the interstitial volume using pressurized nitrogen gas at the end of the run to minimize losses; this column hold up must be processed through a fresh column. This was conducted on a 10 cm long, 1.3 cm diameter glass column, and was later scaled to a larger column (29 cm long, 2.5 cm diameter).

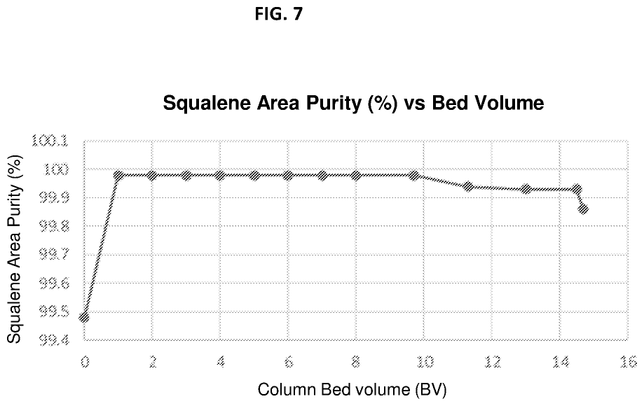

During the larger column trials, a column was dry packed with 138.5 g of basic alumina (2.5×29 cm, 142 mL) and distilled squalene (1.4 kg, 1.63 L) was loaded on to the column at a flow rate of 5 mL/min (1 cm/min superficial velocity) for the first bed volume and increased to 11 mL/min (2.2 cm/min superficial velocity) for the remainder of the run (˜10-minute residence time). A fraction size of 1 Bed Volume (BV), including 142 mL, was collected. Individual fractions were analyzed for impurity breakthrough (FIG. 7) and combined based on GC-FID purity analyses (EP method). The combined BVs that made up the main product stream achieved the squalene weight purity target of >99.5% by weight.

The yield of this alumina column step can be increased with common processing techniques such as draining the column, when the resin is saturated with impurities, using an inert gas (N₂) and then processing the drained oil through a fresh alumina column. Table 7 compares the 3 large column runs that were performed and shows the pressure drop that occurred at two bed lengths (15 cm and 29 cm), as well as the step yield and resulting final oil purity.

TABLE 7
Alumina column parameters and results for 3 lab scale runs

| | Column | Bed | Bed | | BV before | Product |
|---|---|---|---|---|---|---|
| Column | ID | height | Volume | Pressure | impurity | yield |
| run | (cm) | (cm) | (mL) | (bar) | breakthrough | (%) |
| 1 | 2.5 | 15 | 238 | 1 | 9 | 90 |
| 2 | 2.5 | 29.5 | 145 | 2.8 | 9.6 | 91 |
| 3 | 2.5 | 29 | 142 | 2.5 | 9.9 | 90 |

Following the alumina column step, Vitamin E (500 ppm) was added to the purified oil to stabilize the squalene against oxidation. Those of skill in the art will appreciate that any other suitable antioxidant may also be used.

Taken together, the results of the experiments conducted in this example demonstrate that a multi-step purification process that includes extraction, distillation, and chromatographic purification results in the production of high-purity (>99.5% (w/w)) squalene from fermented host cells expressing the enzymes involved in squalene biosynthesis.

### Example 2. Purification of Squalene from Fermentation Broth Using a Two-Part Extraction

In an exemplary purification procedure of the disclosure, squalene is produced by fermentation of yeast cells, and is subsequently purified by homogenization of the cell broth. This is followed by centrifugation, distilling the squalene resulting after homogenization and centrifugation, and purifying the squalene resulting from distillation using chromatography.

Specifically, in some embodiments of the disclosure, whole-cell broth from the squalene fermentation may undergo homogenization at ambient temperature, where the fermentation composition is passed through a homogenizer two times. The homogenization may be carried out at a pressure of between 800 bar and 1,000 bar and at a native pH.

Following homogenization of the whole-cell broth, the resulting homogenate may be centrifuged to remove sedimentable solids and recovered an aqueous-emulsion liquid phase. Prior to centrifugation, the homogenate may be heated to a temperature of about to 60° C. to about 70° C. The homogenate may then be centrifuged at a rate of between 3,000 rpm and 5,000 rpm for a period of between 5 minutes and 30 minutes. After centrifugation, the solids are discarded as waste, and the supernatant is carried on to distillation.

At this stage, an evaporator may be heated to an average temperature of between 150° C. and 250° C., and the supernatant may then be fed into the evaporator for distillation. The distillate stream may then be cooled to ≤70° C. while under vacuum, and the condensed liquid may then be collected.

Using this exemplary process, squalene is further purified by way of chromatography, where the solution containing squalene is run over a fixed bed of basic aluminum oxide at ambient temperature. The particle size of the resin that is used may be between 50 μm and 700 μm, and the flow rate of the column may be between 2 mL/min and 15 mL/min.

### Example 3. Purification of Squalene from Fermentation Broth Using a Three-Part Extraction

In another exemplary purification procedure of the disclosure, squalene is produced using fermentation of yeast cells, and purified by homogenization of the cell broth. This is followed by centrifugation, demulsification of the supernatant obtained from centrifugation, distilling the squalene resulting demulsification of the supernatant, and purifying the squalene resulting from distillation by way of chromatography.

In this example, whole-cell broth from the squalene fermentation undergoes homogenization at ambient temperature, where the fermentation composition is passed through a homogenizer two times. The homogenization occurs at a pressure of between 800 bar and 1,000 bar and at a native pH.

Following homogenization of the whole-cell broth, the resulting homogenate is centrifuged to remove sedimentable solids and recovered an aqueous-emulsion liquid phase. Prior to centrifugation, the homogenate may be heated to a temperature of about to 60° C. to about 70° C. The homogenate is then centrifuged at a rate of between 3,000 rpm and 5,000 rpm for a period of between 5 minutes and 30 minutes. After centrifugation, the solids are discarded as waste, and the supernatant is carried on to be demulsified.

The centrifuged supernatant is mixed with a surfactant, which has a final concentration of between 0.01% and 5% (v/v). The surfactant may be DOWFAX® 2A1, DOWFAX® 3B2, DOWFAX® 8390, DOWFAX® C6L, DOWFAX® C10L, TRITON® QS-15, TRITON® XN-45S, or TERGITOL® L62. The demulsification is performed at a temperature of between 50° C. and 90° C. and at a pH of between 6 and 8. The demulsified supernatant is then further processed using distillation.

For distillation, the evaporator is heated to an average temperature of between 150° C. and 250° C., and the demulsified supernatant is fed into the evaporator. The distillate stream is cooled to <70° C. while under vacuum and the condensed liquid is collected.

Squalene is further purified using chromatography, where the solution containing squalene is run over a fixed bed of basic aluminum oxide at ambient temperature. The particle size of the resin that is used is between 50 μm and 700 μm, and the flow rate of the column is between 2 mL/min and 15 mL/min.

### Example 4. Purification of Squalene from Fermentation Broth Using a Four-Part Extraction

In another exemplary process, squalene may be produced by fermentation of yeast cells, and then purified by extracting the squalene from the fermentation composition. In this example, the extracting includes homogenizing the fermentation composition, separating the resulting homogenized fermentation composition into sediment and supernatant by way of centrifugation, demulsifying the supernatant obtained from centrifugation, and separating the resulting demulsified supernatant into an aqueous component and an oil component. The extracted squalene may then be distilled and purified by way of chromatography.

In this example, whole-cell broth from yeast fermentation may undergo homogenization at ambient temperature. As part of the homogenization process, the fermentation composition may be passed through a homogenizer (e.g., one or more times). In this example, the homogenization occurs at a pressure of between 800 bar and 1,000 bar and at a native pH.

Following homogenization of the whole-cell broth, the resulting homogenate is centrifuged to remove sedimentable solids and recovered an aqueous-emulsion liquid phase. Prior to centrifugation, the homogenate may be heated to a temperature of about to 60° C. to about 70° C. The homogenate is then centrifuged at a rate of between 3,000 rpm and 5,000 rpm for a period of between 5 minutes and 30 minutes. After centrifugation, the solids are discarded as waste, and the supernatant is carried on to be demulsified.

The centrifuged supernatant is mixed with a surfactant, which has a final concentration of between 0.01% and 5% (v/v). The surfactant may be DOWFAX® 2A1, DOWFAX® 3B2, DOWFAX® 8390, DOWFAX® C6L, DOWFAX® C10L, TRITON® QS-15, TRITON® XN-45S, or TERGITOL® L62. The demulsificaiton is performed at a temperature of between 50° C. and 90° C. and at a pH of between 6 and 8. The demulsified supernatant is then further processed using liquid-liquid centrifugation. The liquid-liquid centrifugation is performed at a temperature of between 50° C. and 90° C., at a rate of between about 3,000 rpm to about 5,000 rpm, and for a period of between 5 minutes to 30 minutes.

Following liquid-liquid centrifugation the resulting oil component is mixed with an aqueous base solution, where the base may be NaOH, LiOH, KOH, and Ca(OH)₂. The base solution may have concentration of about 1 M hydroxide and a pH of between 9 and 13. The oil component form from the liquid-liquid centrifugation is mixed with the basic solution at a ratio of about 0.5:1 (v/v). The mixing of the basic solution with the oil component occurs at a temperature of between 20° C. and 80° C. and for a time period of between 30 minutes and 2 hours. Following washing the oil component with the basic solution, the squalene is purified by distillation.

For distillation, the evaporator is heated to an average temperature of between 150° C. and 250° C., and the squalene containing solution is fed into the evaporator. The distillate stream is cooled to ≤70° C. while under vacuum and the condensed liquid is collected.

Squalene is further purified using chromatography, where the solution containing squalene is run over a fixed bed of basic aluminum oxide at ambient temperature. The particle size of the resin that is used is between 50 μm and 700 μm, and the flow rate of the column is between 2 mL/min and 15 mL/min.

### Example 5. Investigation of Temperature and Pressure During Distillation on Purity

The goal of this example was to evaluate the effect of temperature (C), system vacuum pressure (torr), condenser temperature (° C.) and feed flow rate (L/h/m²) and responses distillate purity (area %, GC-FID), impurity concentration (area %, GC-FID), distillate to feed ratio [D:F] have on yield (%).

The impact of temperature (still-body temperature), pressure, feed flow-rate and condenser temperature on the CQAs purity (area %) and impurity concentrations (area %) were investigated. Process performance was also evaluated by D:F and yield by gain to determine feasibility of factor levels.

The range of still-body temperature studied was from 200 to 250° C. The pressure limits were set at 0.7 torr for the minimum because this is the minimum attainable pressure across the system at full vacuum and at 2 torr at the high end to account for deviations in vacuum that would occur at scale.

The feed flow rates tested were 30 to 48 L/h/m², normalized by evaporator area to compare across scales. This range was chosen because the minimum operating flow rate attainable at scale is 30 L/h/m² while the maximum operating flow rate achievable using the 4″ WFE is 48 L/h/m².

The condenser temperature was varied from 20 to 60° C. The minimum condenser temperature in this range was chosen due to system cooling limitations and considering distillate viscosity at low temperatures. The maximum condenser temperature was chosen from the previous bench-scale operating set-point used on the 2″ glass WFE.

Factors investigated included temperature, pressure, feed flow rate and condenser temperature with levels listed in Table 8. The responses measured were distillate purity, impurity concentration, D:F, and yield.

TABLE 8
Summary of experimental design and run conditions

| | Temperature | Pressure | Feed Flow Rate | Condenser |
|---|---|---|---|---|
| Run | (° C.) | (torr) | (L/h/m\hat{ }2) | Temperature (° C.) |
| 1 | 200 | 2 | 30 | 20 |
| 2 | 225 | 2 | 30 | 40 |
| 3 | 200 | 0.7 | 30 | 60 |
| 4 | 225 | 0.7 | 30 | 60 |
| 5 | 225 | 1 | 39 | 60 |
| 6 | 250 | 1 | 39 | 60 |
| 7 | 200 | 0.7 | 48 | 60 |
| 8 | 225 | 1 | 48 | 40 |
| 9 | 250 | 0.7 | 48 | 40 |
| 10 | 250 | 2 | 30 | 20 |
| 11 | 250 | 1 | 39 | 20 |

The mass of 250 mL round bottom flasks were recorded. 4 L of Polished Crude Oil was added to the feed tank of WFE. The heat feed tank was set to 70-80° C., and heat tape was turned on to insulate feed line to WFE still body. The 250 ml round bottom flask (RBF) was attached to the cold trap receiver. The cold trap was filled up to half-way with Isopropanol and slowly dry ice was added. The vacuum pressure was set to desired level for run as listed in Table 8. The jacket temperature was set to the desired level for the run as listed in Table 8. The condenser temperature was set to the desired level for the run as listed in Table 8. Using the pump calibration curve, the corresponding RPM on pump controller was set for designated run flowrate as listed in Table 8. The wipers were set to 60-70% power. The temperature was monitored and readout to observe when steady state has been achieved. When temperature readouts reached a steady state, the wipers and feed pump were paused. The isolation valve on glass adaptors was closed to receiving RBFs on both Distillate and Bottoms lines. Venting valves on glass adaptors were opened to break vacuum to receiving RBFs. Receiving RBFs were removed once the vacuum was broken and replaced with clean, pre-weighed 250 ml RBFs. Venting valves on adaptors were closed and adaptor isolation valves were opened and were allowed to evacuate. The feed pump was turned on followed by wiper drive and was allowed to collect for 3 minutes. While collecting in 250 ml RBFs, 3 L RBFs were emptied into one 5 L container. The process was repeated for each run until the feed tank was close to empty.

The Crude Squalene Oil feed used for this experiment had an initial squalene concentration of 82.5 wt %. A summary of the typical feed composition ranges are shown in Table 9.

TABLE 9
Summary of typical Crude Squalene
Oil used as feed to evaporation

| | | Dynamic | Sedimentable |
|---|---|---|---|
| Squalene | Density | viscosity | solids |
| (wt %) | (g/mL, @20° C.) | (cP, @20° C.) | (% vol) |
| 75-85 | 0.87-0.88 | 20-25 | <0.1 |

Experimental raw data for distillate purity (% area) and impurities (area %) measurements and yield calculations are shown in below in Table 10.

TABLE 10
Summary of experimental raw data for runs shown in Table 9

| | Purity | [Impurities] | D:F | Yield by |
|---|---|---|---|---|
| Run | (Area %) | (Area %) | (% w/w) | Gain (%) |
| 1 | 92.5 | 8.2 | 1.8 | 1.9 ± 0.1 |
| 2 | 98.5 | 1.6 | 11.4 | 13.6 ± 0.6 |
| 3 | 98.2 | 2.0 | 11.4 | 13.5 ± 0.0 |
| 4 | 99.4 | 0.7 | 67.7 | 78.7 ± 1.0 |
| 5 | 99.4 | 0.7 | 52.2 | 63.0 ± 0.8 |
| 6 | 99.2 | 0.8 | 75.2 | 93.0 ± 1.2 |
| 7 | 97.3 | 3.0 | 4.9 | 3.0 ± 2.9 |
| 8 | 99.0 | 1.1 | 39.9 | 50.0 ± 0.3 |
| 9 | 98.9 | 1.1 | 75.7 | 92.2 ± 0.1 |
| 10 | 99.3 | 1.0 | 69.5 | 85.6 ± 0.1 |
| 11 | 99.0 | 0.8 | 60.4 | 72.5 ± 1.4 |

Distillate purity was measured using Area % analysis. Linear regression analysis was performed on JMP; statistical significance of the observed operating parameters on distillate purity was determined by calculating the p-values for the variable effects on distillate purity using a 95% confidence interval. P-values are the probability of obtaining a more extreme change in purity given that the null hypothesis is true—the variable has no effect on purity; thus, the lower the p-value the greater the significance.

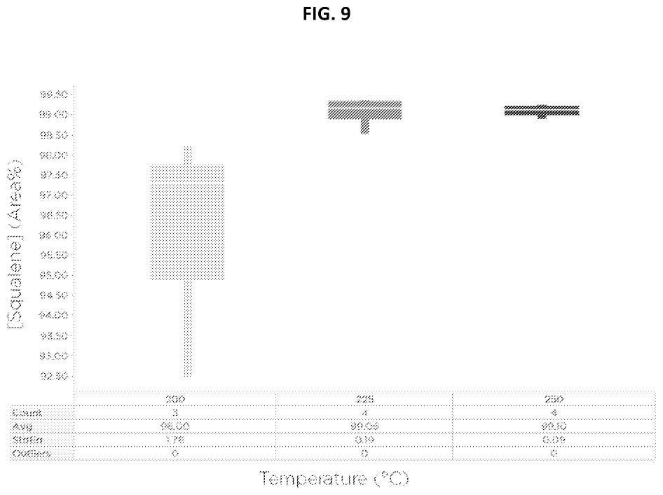

Pressure, feed flowrate, and condenser temperature did not have significant effect on distillate purity as their p-values are above 0.05. The only significant parameter at a 95% confidence interval was temperature, where the p-value was 0.049. The impact of temperature on distillate purity is shown in FIG. 9, which highlights that the impact of temperature was primarily at lower temperatures 200-225° C. for the pressures tested, and there was likely a threshold temperature between these where temperature no longer impacts purity.

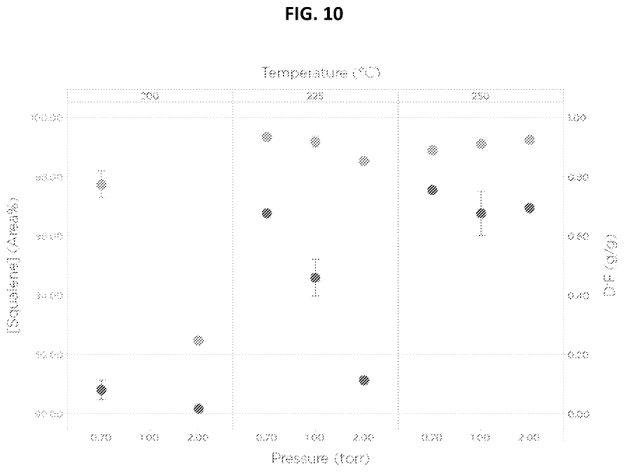

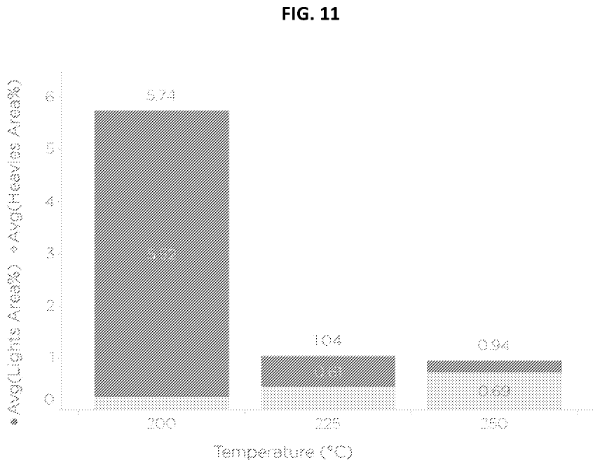

The impact of the combination of temperature and pressure on distillate purity is further shown in FIG. 10 by binning the data according to temperature and plotting pressure on x-axis of each bin. FIG. 10 also shows the distillate to feed ratio (D:F) which provides an indication of the amount of squalene evaporated and was a key in-process indicator of performance. Lower purity was seen at low temperature and low pressure (200° C., 2 torr) and also coincides with low D:Fs (<10%); these results indicated incomplete evaporation of squalene into the distillate, resulting in higher concentrations of lighter impurities, including fatty acids (FAs) and fatty esters, and lower concentration of product and heavy impurities (FIG. 11).

A one-way analysis of variance (ANOVA) was performed using JMP to determine if there was a statistically significant difference between the purities produced at the different temperatures. Using a 95% confidence interval, p-values for the differences in purity between temperatures below 0.05 indicate that the difference in purity is significant. The One-way ANOVA confirmed the purities produced at 200° C. were significantly lower than both higher temperature conditions while there was no significant difference between those produced at 225 and 250° C., indicating that distillate purity is stable at this temperature range.

Though the highest distillate purity was produced between 225 and 250° C. and remained constant within this temperature range, the concentration and composition of impurities must be considered as it will affect the performance of the alumina column treatment. The amount of feed that can be processed on the column before impurities begin to break through into the final product at an unacceptable concentration (column capacity) were dependent on the concentration of the impurities. Column capacity was also impacted by the type of impurities within the distillate as some species may have lower affinity to the alumina resin and may break through sooner, reducing the capacity.

FIG. 11 shows the average impurity concentration (area %) for each temperature as stacked bars denoting the light impurities (those that elute on the GC-FID method before squalene), including FAs and FA esters, and heavy impurities (those that elute on the GC-FID method after squalene), such as sterols. The greatest concentration of light impurities was observed during the 200° C. conditions (5.52% area) because of low squalene carry-over indicated by lower D:F. As temperature was increased, the concentration of lights decreased while the concentration of heavy impurities increased with the highest concentration at 250° C. (0.69% area). Though heavy impurities increased as temperature increased, the overall concentration of impurities was lower in the higher temperature range due to increased squalene evaporation. The total impurity concentration did not differ significantly between 225 and 250° C. (1.04 and 0.94%, respectively).

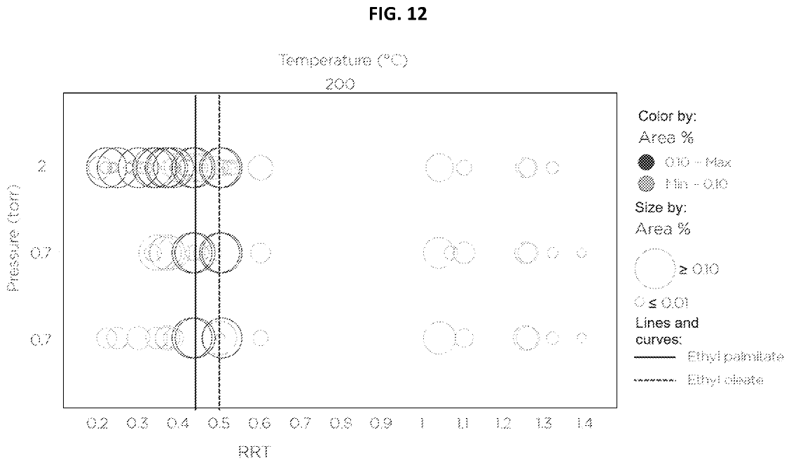

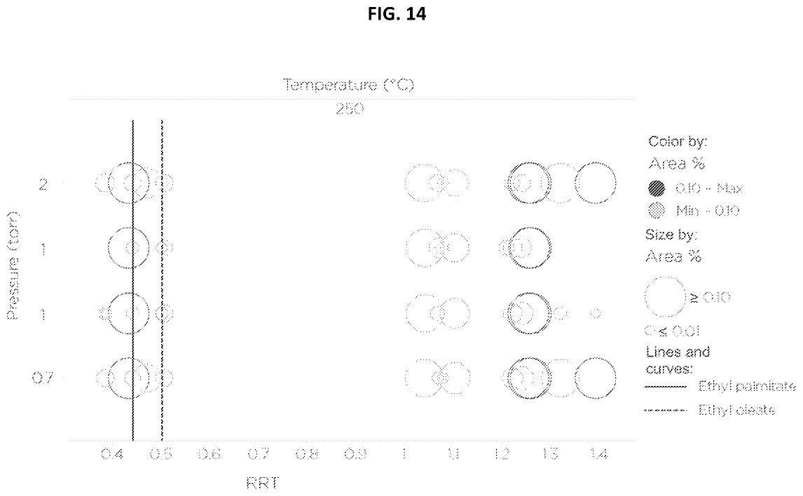

FIGS. 12-14 show bubble plots of the relative area % profile of non-squalene peaks detected in each distillate by their relative retention time (RRT) to the main squalene peak. Each bubble represents a detected species in the distillate and the size of each bubble increased as the concentration in area % increased. The smallest bubbles represent concentrations below 0.01% while the largest bubbles in red represent concentrations above 0.10%, the concentration at which identification of the species was required in the final material. Known RRTs for impurity peaks ethyl palmitate and ethyl oleate were labelled by solid and dotted lines, respectively.

FIG. 12 shows that the distillates produced at 200° C. contained a greater number of light impurities in the early RRT region below 1 RRT than heavy impurities above 1 RRT with some light impurities and no heavy impurities that exceeded 0.10%. The distillate that contained the largest number of non-squalene peaks was produced at 200° C. and 2 torr and also contained the highest number of peaks that exceeded 0.10%. These results were consistent with poor squalene recovery in the distillate, leading to a greater concentration of light non-squalene impurities. Because of the lower purities and D:Fs (<20% shown in FIG. 10).

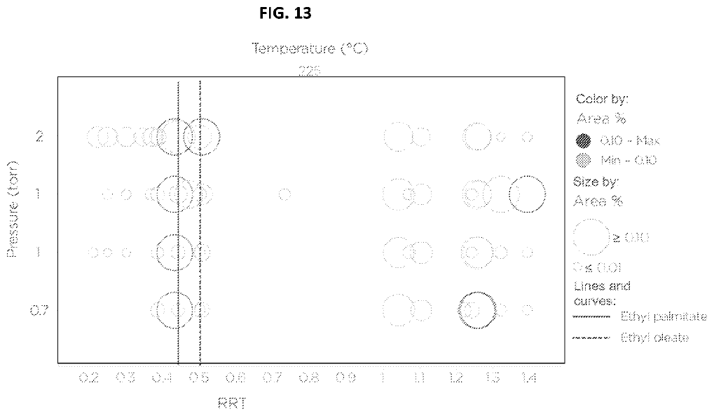

In the 225° C. distillates (FIG. 13), the higher concentration of squalene reduced the relative area % of peaks within the lights region compared to the 200° C. distillates. At this higher temperature, however, there were a greater number of peaks within the heavies region across the different pressures tested. There was one species within 0.4-0.5 RRT that was above 0.10%, and while there were a greater number of peaks in the heavies region at 225° C., most peak concentrations are below 0.10%.

FIG. 6 shows that the distillates produced at 250° C. have fewer peaks in the lights region than either 200 or 225° C. (FIGS. 4 and 5, respectively), while having a similar number of peaks within the heavies region as 225° C. At 250° C., pressure does not significantly impact the number of impurity peaks in the distillate. The peak between 0.4 and 0.5 RRT seen in 225° C. is still present in the distillates produced at 250° C. and is greater than 0.10%. There are peaks within the heavies region that began to appear at 225° C. are now above 0.10% at 250° C.

One peak of interest within these distillates between 1.0 and 1.1 RRT that was shown to not be affected by alumina column treatment remains at about 0.09% but will continue to be monitored. The increase in number of peaks in the heaviest region is potentially a result of greater distillation of heavy boilers but also results in greater D:Fs and yield.

Yield was calculated using concentrations of squalene (wt %) within the sample streams measured by the appropriate Megalodon DSP titer assays. Assuming no losses in the system, the mass of the feed was estimated to be the sum of the mass of the collected distillate and bottoms. Yield was then calculated using the following equation:

$$Yield = \frac{{[ Squalene ]}_{distillate} * {m}_{distillate}}{{[ Squalene ]}_{feed} * ( {m}_{distillate} + {m}_{bottoms} )}$$

Where,
[Squalene]_{distillate}=Concentration of squalene in distillate (g/kg)
[Squalene]_{feed}=Concentration of squalene in feed (g/kg)
m_{distillate}=Mass of distillate (g)
m_{bottoms}=Mass of bottoms (g)

Linear regression was performed on JMP to determine the statistical significance of the CPPs on yield. Significance was determined by calculating the p-values for the variable effects on yield using a 95% confidence interval. Temperature and pressure were determined as CPPs with p-values of 0.00, whereas feed rate and condenser temperature were not significant with p-values greater than 0.05.

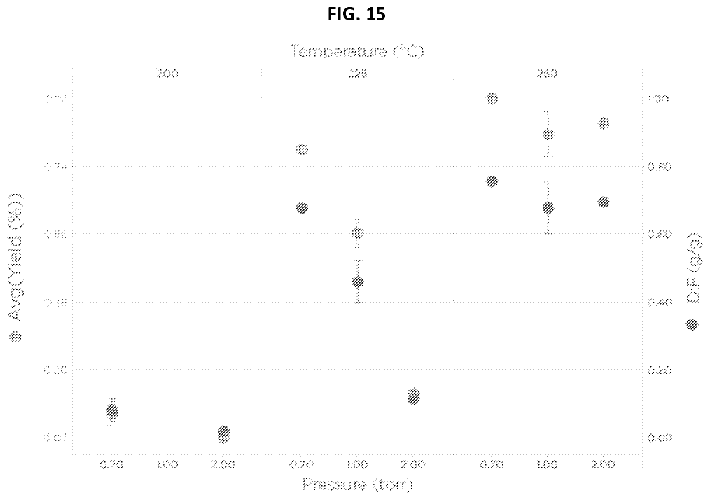

Average yields along with the measured D:Fs are plotted in FIG. 15 by temperature and pressure. The highest yield of about 92.0% was achieved at 250° C. and 0.7 torr, whereas the lowest yield of about 2.0% was observed at 200° C. and 2 torr.

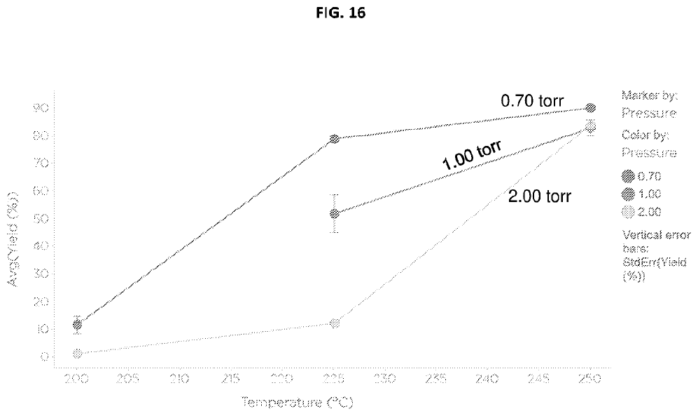

The magnitude in which yield changed by pressure varied between temperature levels, so a variable interaction between pressure and temperature was also investigated in the linear regression. The p-value for this interaction was 0.00, indicating that the effect of temperature and pressure on yield is dependent on one another. This interaction is illustrated in FIG. 16, where the average yields by temperature for each operating pressure were plotted. Here the change in yield by pressure was shown to be dependent on the operating temperature of the system, where the greatest change in yield by pressure was experienced at 225° C. This behavior was consistent with the model evaporation system where at low temperatures the product did not evaporate at any of the test pressures, at some threshold temperature the product evaporated at the lower pressures but not at the higher pressures showed a strong pressure dependence, and then at higher temperatures the product evaporated at all pressures tested.

The average yields at 200° C. are low (<15%) all pressures tested, indicating this temperature is below the PAR needed for adequate squalene recovery and confirms these conditions are at the EoF. At 225° C., average yields decrease from 80% to 10% as system pressure is increased from 0.7 to 2 torr, while the average yields at 250° C. remain above 80% across all pressures. This suggests the PAR for temperature begins at 225° C. where satisfactory squalene recovery occurs at vacuums below 1 torr. Because yield losses due to fluctuations in pressure are minimized at 250° C. without a significant change in purity, this temperature level is considered the optimal set point for evaporation at any operating pressure from 0.7 to 2 torr.

In order to confirm the results seen in the design of experiments (DOE), 4 L engineering runs were performed using the higher operating set temperature (250° C.). An engineering run at the maximum system operating temperature of the 4″ WFE (255° C.) was also performed to further test the upper limit on the PAR for temperature. The operating pressure for these runs was held at 1 torr while the feed flow rate and condenser temperature were 39 L/h/m² and 60° C., respectively. A summary of the results is found in Table 11 along with a summary of the DOE run at the same condition for comparison.

TABLE 11
Run summary of 250 and 255° C. evaporations

| | | | | | [Light | [Heavy |
|---|---|---|---|---|---|---|
| | | | | | Impu- | Impu- |
| | Temp | D:F | [Squalene] | Yield | rities] | rities] |
| Scale | (° C.) | (g/g) | (area %) | (%) | (area %) | (area %) |
| DOE | 250 | 72.4 | 99.2 | 93.0 ± 1.2 | 0.12 | 0.59 |
| 4 L | 250 | 81.1 | 99.3 ± 0.01 | 96.6 ± 0.5 | 0.14 | 0.63 |
| | 255 | 81.9 | 99.2 | 96.6 ± 0.6 | 0.14 | 0.69 |

The distillate purity was ≥99.2% area and the recovery remained high with yields at about 97% at both temperatures, similar to the smaller scale DOE run at the same conditions. These results confirmed the observations made during the DOE and indicate that the PAR for temperature can be extended to include 255° C. with no negative impact to purity or yield.

### Conclusions

Temperature and pressure had a significant impact on purity and concentration of impurities. At lower temperatures (200° C.), poor squalene distillation led to higher concentrations of FAs in the distillate and lower squalene purity. At higher temperatures (225-250° C.), the distillate produced was >99.0% area squalene though there were a greater concentration of heavies present.

Yield increased as temperature increased from 200 to 250° C. However, the loss in yield was a result of increases in pressure were dependent on temperature. At the low end of the PAR for temperature (225° C.), yield was negatively impacted by increases in system pressure. The yield loss due to pressure fluctuations can be negated at the set point 250° C. which experienced no significant change in yield due to pressure increase. The engineering runs at 250 and 255° C. confirmed the results seen during the DOE and demonstrate that the PAR can be expanded up to 255° C.

### Example 6. Effect of Alumina Polishing on Purity

The experiment was performed to determine the effect of alumina polishing on squalene purity. Factors investigated include alumina moisture content as measured by loss on drying (LOD), residence time of squalene on the column, and breakthrough (≥0.1%) of specific impurities at specific bed volumes (BVs). Squalene area % purity was also tracked, but because in all the distillates used it was already within the final specification (≥99.0%) it was seen as less important than impurities within the feed that would be the first to break through the column and be projected to be the impurity to first reach the ≥0.1%. These were labeled as “critical impurity RRT” (Table 12). The critical impurity differed from feed to feed depending on fermentation performance and processing prior to alumina polishing. Breakthrough of the critical impurity was influenced by the number and amount of other impurities present in the distillate.

TABLE 12
Summary of experimental factors based on alumina column conditions,
distillate purity and critical impurity identification

| | Alumina | | | | critical impurity | GC area % |
|---|---|---|---|---|---|---|
| | Moisture | Residence | | GC area % | RRT (1^{st} to | of critical |
| Experiment | % by | time | Distillate | purity of | breakthrough | impurity in |
| ID | LOD | (min) | ID | distillate | alumina) | distillate |
| 1172-60 | 1.3 | 24 | 1172-60- | 99.23 | 1.07 | 0.1184 |
| | | | Blend | | | |
| | | | Feed | | | |
| 1172-61 | 2.1 | 24 | 1172-60- | 99.23 | 1.07 | 0.1184 |
| | | | Blend | | | |
| | | | Feed | | | |
| 1172-62 | 3.9 | 24 | 1172-60- | 99.23 | 1.07 | 0.1184 |
| | | | Blend | | | |
| | | | Feed | | | |
| 1172-64 | 2.4 | 12 | H11926 | 99.39 | 0.427 | 0.127 |
| | | | Dist | | | |
| | | | 05182022 | | | |
| 1172-63 | 2.4 | 24 | H11926 | 99.39 | 0.427 | 0.127 |
| | | | Dist | | | |
| | | | 05182022 | | | |
| 1172-65 | 2.4 | 36 | H11926 | 99.39 | 0.427 | 0.127 |
| | | | Dist | | | |
| | | | 05182022 | | | |
| 1172-67 | 2.5 | 24 | H11954 | 99.20 | 1.252 | 0.193 |
| | | | Dist 250C | | | |
| 1172-68 | 2.3 | 24 | H11954 | 99.37 | 1.252 | 0.212 |
| | | | Dist 255C | | | |

Dry granular basic alumina was placed in the oven at 150° C. for six to twelve hours to bring moisture down to below 2% as measured by LOD on moisture balance (160° C. until no change in mass). ˜82 grams of dried granular alumina was placed into 2.5 cm ID glass column to 30 cm height tamping as loading to help alumina settle or pack the column bed. Bed volume (BV) in milliliters was calculated by BV=π×r²×height. BV of 147.2 ml (126.3 g) was collected for each experiment. 40-70 mg of alpha tocopherol (vitamin E) was placed in 6-12 250 ml amber bottles. To run the column out to 12 BVs, 896 ml (1627 g) of distilled squalene was placed in a 2 liter amber bottle and 75-80% of a BV was assumed as a loss to column (blowdown+hold-up). With the feed line detached from column, ˜5 grams of distillate was run through the pump and feed line to clean it, and squalene was discarded. The feed line was then attached to the column. The pump was started at approximately 35% power (which corresponded to 24 min residence time) to wet the column with squalene. The first bottle to collect the first BV under column was placed on the balance and zeroed. 126.3 g (147.2 ml) squalene was collected in bottle, and the duration for collection and pressure on the gauge were noted. These steps were repeated until all BVs were collected. Bottles were shaken to mix in the vitamin E already present. The pump was stopped, and nitrogen was used on top of the column to blow down for 30 minutes and collect the mass of squalene (blow-down). The spent alumina was discharged, and the mass collected. For losses of squalene, the alumina mass was subtracted from the discharge mass, and blow-down mass was added. For storage of BVs, the bottles were charged with nitrogen in the headspace and stored at 5° C.

GC area % data was used to track impurities through each BV collected during the running of the columns and plotted to show which impurity broke through the ≥0.1%, this was considered the critical impurity for that particular distillate. The results for the eight columns run with four different distillates was summarized in Tables 12 and 13.

TABLE 13
Summary of experimental results including critical impurity breakthrough

| | | | | | Predicted |
|---|---|---|---|---|---|
| | | | Critical | | alumina |
| Alumina | | Predicted | impurity RRT | GC area % | breakthrough |
| Moisture | | GC area % | (1^{st} to | of critical | (≥0.1%) of |
| % by | Residence | purity of | breakthrough | impurity in | critical impurity |
| LOD | time (min) | BV1-5 | alumina) | distillate | (BV) |
| 1.3 | 24 | 99.66 | 1.07 | 0.1184 | 11 |
| 2.1 | 24 | 99.68 | 1.07 | 0.1184 | 10 |
| 3.9 | 24 | 99.58 | 1.07 | 0.1184 | 6 |
| 2.4 | 12 | 99.83 | 0.427 | 0.127 | 9 |
| 2.4 | 24 | 99.86 | 0.427 | 0.127 | 10 |
| 2.4 | 36 | 99.86 | 0.427 | 0.127 | 10 |
| 2.5 | 24 | 99.72 | 1.252 | 0.193 | 15 |
| 2.3 | 24 | 99.85 | 1.252 | 0.212 | 13 |

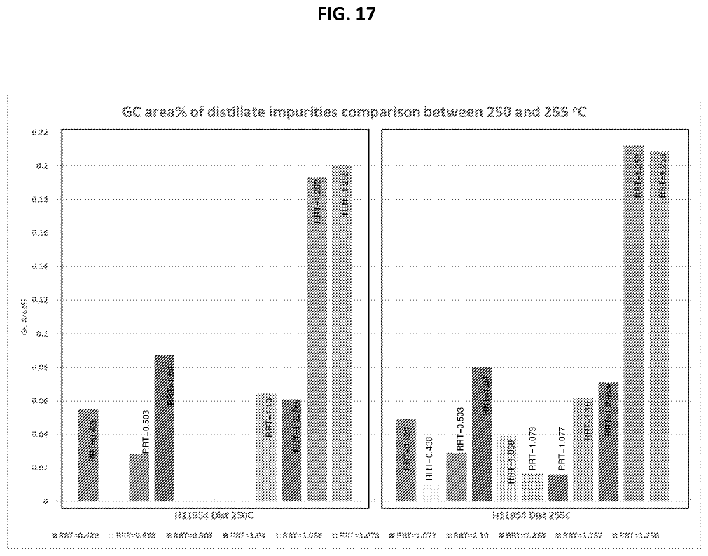

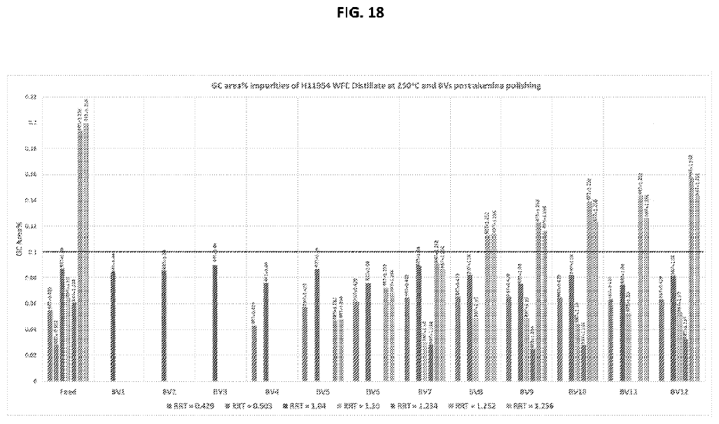

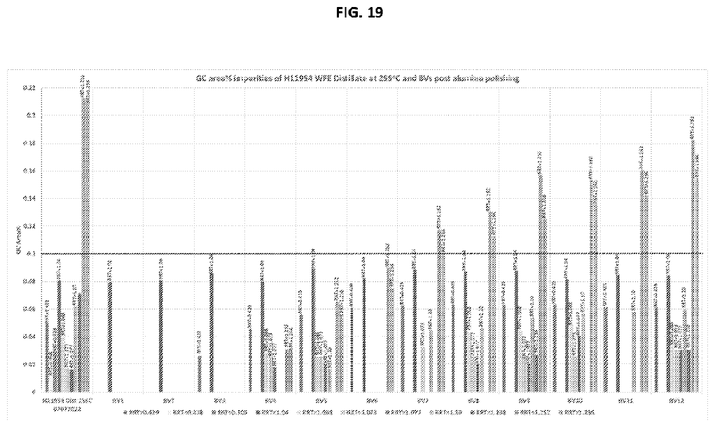

Two alumina polishing experiments evaluated impurity breakthrough, specifically RRT=1.252, from distillations performed at 250 and 255° C. Higher levels of and additional impurities were observed in the squalene distilled at 255° C. (FIG. 17). This increase in impurities reduced the number of BVs collected within the GC area % specification of ≤0.1%. These two experiments were run out to 12 BVs instead of the six or eight. The distillates with impurity levels and all 12 BVs are shown in FIGS. 18 and 19.

From these two charts, it was observed that the breakthrough of the two sterols RRT=1.252 and 1.256 happened earlier (BV7) in the 255° C. distillate than in the 250° C. (BV8). It should be noted that these two sterols are seen at similar levels in the feed and behave similarly during polishing, with RRT=1.252 being slightly earlier to breakthrough. Both sterols were considered critical impurities in these feeds.

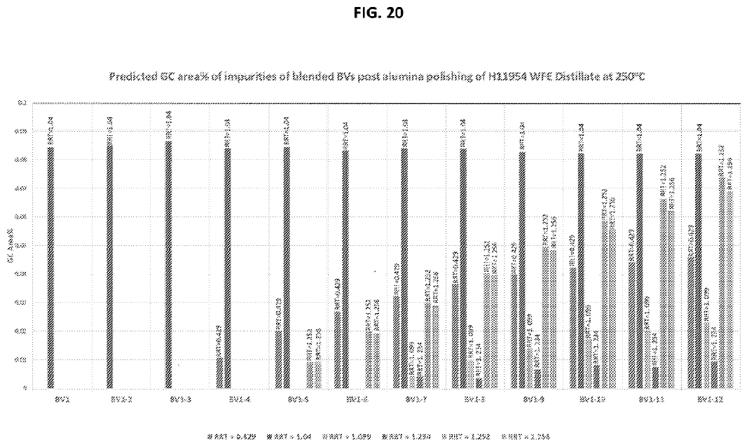

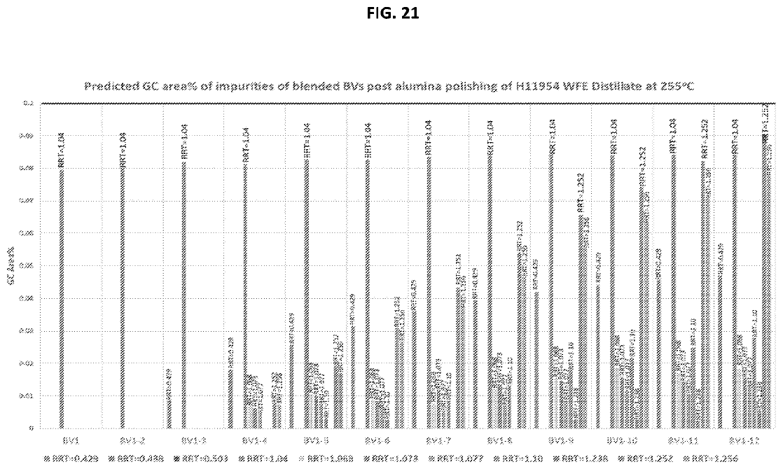

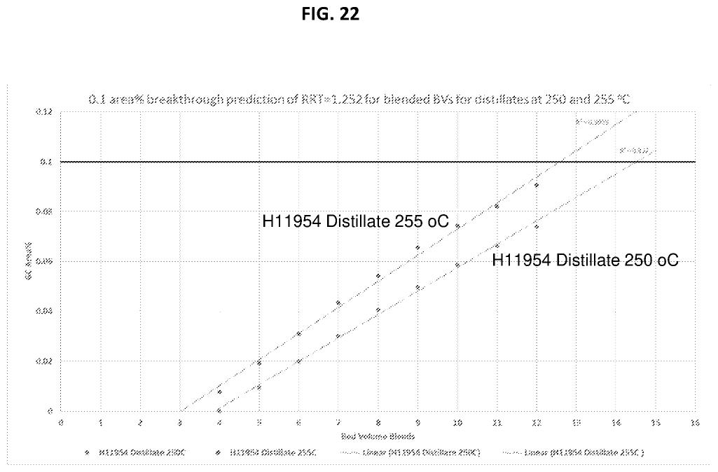

Because bed volumes were blended, the final pooled product impurities were diluted by earlier BVs. A prediction can be made by averaging the GC area % of the BVs blended out as far as the twelve BVs collected. This provided a reasonable assessment of the impurity concentrations at each level of blending (FIGS. 20 and 21). The predicted results showed that all twelve BVs could be blended for polished squalene from both the 250 and 255° C. distillates. By plotting the GC area % of RRT=1.252 against the blends and then extrapolating, a prediction for breakthrough was made (FIG. 22). This resulted in a predicted breakthrough of this sterol at BV 13 with the 255° C. distillate whereas the polished squalene using distillate at 250° C. had a predicted breakthrough at BV 15.

### Conclusions

Higher levels of moisture adsorbed to the basic alumina which significantly decreased the removal of impurities. The longer the residence time of the squalene on the alumina bed, the better it was for the removal of impurities. Because the concentration of the impurity RRT=1.04 was not reduced by the alumina, it was important to confirm that it is below 0.1% in the distillate prior to loading the column. The fatty ester impurity at RRT=0.429 (ethyl-9-hexadecenoate) had less affinity for the alumina than the sterols in general and could be a critical impurity if distillation temperature was lower than planned. Distillation at higher temperature (≥225-255° C.) reduced this impurity and increased the sterol levels. Critical impurities RRT=1.07, RRT=1.252 and RRT=1.256 were most likely to breakthrough the alumina first and be at the highest levels in the final blend.

### Example 7. Purification of Squalene from a Plant Source

Using conventional techniques or techniques described herein, squalene may be extracted from a plant source. Any plant capable of producing squalene or any plant modified so as to be capable of producing squalene may be used. For example, squalene may be extracted from a plant oil such as olive oil, soybean oil, grape seed oil, hazelnut oil, peanut oil, sesame oil, sunflower oil, coriander seed oil, and in corn oil. Squalene may also be extracted from amaranth, rice bran, wheat germ, Monkey Jack, or olives. The squalene may be extracted from a plant source using any conventional technique such as mechanical pression or by using a chemical extraction technique including but not limited to extraction with organic solvents such as hexane.

According to the methods disclosed herein, squalene extracted from a squalene source such as a plant source may be purified by first evaporating the squalene from the extraction composition. The evaporation step may include distilling the squalene. Either fractional distillation or simple distillation may be used. The distillation process may involve, for example, initially heating the squalene to a temperature of from about 20° C. to about 90° C. For example, the squalene may be initially heated to a temperature of from about 60° C. to about 70° C. The squalene may be evaporated at a temperature of from about 150° C. to about 250° C. For example, the squalene may be evaporated at a temperature of from about 200° C. to about 205° C. Evaporation of the squalene may be performed under vacuum. For example, the evaporation may be performed at a pressure of about 0.5 to about 5 torr, including pressures between about 0.7 torr and 4 torr, 0.7 torr and 2 torr, or 2 torr and 4 torr.

Following evaporation of the squalene, the squalene may be condensed and cooled. Condensation and cooling of the squalene may be performed at a temperature of about 70° C. or less, such as a temperature of from about 20° C. to about 70° C. For example, the squalene may be condensed and cooled at a temperature of from about 20° C. to about 25° C. Condensation and cooling of the squalene during evaporation may be performed under vacuum. For example, in some instances, the evaporation may be performed at a pressure of about 1 torr. The evaporation of the squalene may be performed using a wiped film evaporator.

Following evaporation of the squalene from the extraction composition, the squalene is then purified by way of chromatography. Chromatography may be used to further isolate squalene. The chromatography step may include, for example, exposing the squalene to a resin (e.g., a polar resin) and recovering squalene from the resin. The resin may be aluminum oxide resin, such as basic aluminum oxide resin, an acidic aluminum oxide resin, or a neutral aluminum oxide resin. In some embodiments, the resin may be a silica resin. The particle size of the resin may be, for example, between about 50 μm to about 700 μm. The resin may require an activation step. The activation step may be, for example, drying of the resin prior to use.

The chromatography may be performed using a flow rate of from about 5 mL/min to about 20 mL/min. For example, the flow rate may be between about 5 mL/min and about 11 mL/min. The flow rate may also be measured in terms of bed volumes, wherein the flow rate is between about 1 bed volumes per hour (BV/hr) to about 5 BV/hr. For example, the chromatography may be performed using a flow rate of from about 1.5 BV/hr to about 3 BV/hr.

The purity of the resulting squalene may be assessed. The concentration of the squalene relative to the total amount of the squalene and the one or more impurities in the composition may be, for example, from 90 wt % to 100 wt % or more, e.g., 90 wt %, 90.5 wt %, 91 wt %, 91.5 wt %, 92 wt %, 92.5 wt %, 93 wt %, 93.5 wt %, 94 wt %, 94.5 wt %, 95 wt %, 95.5 wt %, 96 wt %, 96.5 wt %, 97 wt %, 97.5 wt %, 98 wt %, 98.5 wt %, 99 wt %, 99.5 wt %, 99.9 wt %, or 100 wt %, or more. For example, the concentration of squalene relative to the total amount of the squalene and the one or more impurities in the composition may be from about 99.5 wt % to about 100 wt %, or more, for example, about 99.5 wt %, 99.6 wt %, 99.7 wt %, 99.8 wt %, 99.9 wt %, or 100 wt %.

The squalene concentration relative to the total amount of the squalene and the one or more impurities can be up to 100 wt %, such as up to 100 wt %, 99 wt %, 98 wt %, 97 wt %, 96 wt %, 95 wt %, 94 wt %, 93 wt %, 92 wt %, 91 wt %, or 90 wt %. The squalene concentration relative to the total amount of the squalene and the one or more impurities may be greater than 90 wt %, e.g., greater than 90 wt %, greater than 91 wt %, greater than 92 wt %, greater than 93 wt %, greater than 94 wt %, greater than 95 wt %, greater than 96 wt %, greater than 97 wt %, greater than 98 wt %, greater than 99 wt %, greater than 99.1 wt %, greater than 99.2 wt %, greater than 99.3 wt %, greater than 99.4 wt %, greater than 99.5 wt %, greater than 99.6 wt %, greater than 99.7 wt %, greater than 99.8 wt %, or greater than 99.9 wt %.

The resulting squalene may include one or more impurities, such that the concentration of the one or more impurities relative to the total amount of the squalene and the one or more impurities is from about 0.1 wt % to about 0.5 wt %, such as about 0.1 wt %, 0.2 wt %, 0.3 wt %, 0.4 wt %, or 0.5 wt %. The concentration of the one or more impurities relative to the total amount of the squalene and the one or more impurities may be less than 0.5 wt %, e.g., less than 0.4 wt %, less than 0.3 wt %, less than 0.2 wt %, or less than 0.1 wt %. In some embodiments, the concentration of the one or more impurities relative to the total amount of the squalene and the one or more impurities is about 0.1 wt %, about 0.2 wt %, about 0.3 wt %, about 0.4 wt %, or about 0.5 wt %.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of isolating squalene from a squalene source, the method comprising:
(a) extracting the squalene from the squalene source;
(b) optionally evaporating the squalene resulting from (a); and
(c) purifying the squalene resulting from (b) by way of chromatography.

2. A method of making squalene, the method comprising:
(a) providing a squalene source;
(b) extracting the squalene from the squalene source;
(c) optionally evaporating the squalene resulting from (b); and
(d) purifying the squalene resulting from (c) by way of chromatography.

3. The method of claim 1 or 2, wherein the squalene source is a fermentation source.

4. The method of claim 3, wherein the fermentation source comprises yeast.

5. The method of claim 1 or 2, wherein the squalene source is a plant source.

6. The method of claim 5, wherein the plant source comprises olive, soybean, grape seed, grape, hazelnut, peanut, corn, amaranth, rice, wheat germ, coriander, sesame, or sunflower.

7. The method of claim 1 or 2, wherein the squalene source is an animal source.

8. The method of claim 1 or 2, wherein the squalene source is a fungi source.

9. The method of claim 1 or 2, wherein the fermentation source comprises a stramenopile source.

10. The method of claim 9, wherein the stramenopile source comprises algae.

11. A method of isolating squalene from a fermentation composition that has been produced by culturing a population of host cells capable of producing squalene in a culture medium and under conditions suitable for the host cells to produce squalene, the method comprising:
(b) extracting the squalene from the fermentation composition;
(c) optionally evaporating the squalene resulting from (a); and
(d) purifying the squalene resulting from (b) by way of chromatography.

12. A method of making squalene, the method comprising:
(a) culturing a population of host cells capable of producing squalene in a culture medium and under conditions suitable for the host cells to produce squalene, thereby producing a fermentation composition;
(b) extracting the squalene from the fermentation composition;
(c) optionally evaporating the squalene resulting from (b); and
(d) purifying the squalene resulting from (c) by way of chromatography.

13. The method of claim 11 or 12, wherein the extracting comprises one or more of: homogenization, centrifugation, solvent extraction, and demulsification.

14. The method of any one of claims 11-13, wherein the extracting comprises one or more of:
(i) homogenizing the fermentation composition;
(ii) separating the homogenized fermentation composition resulting from (i) into sediment and supernatant by way of centrifugation;
(iii) demulsifying supernatant obtained from (ii); and
(iv) separating the demulsified supernatant resulting from (iii) into an aqueous component and an oil component.

15. The method of claim 14, wherein the extracting comprises homogenizing the fermentation composition.

16. The method of claim 15, wherein, prior to homogenizing the fermentation composition, the fermentation composition is diluted in water to a final concentration of from about 20% to about 40% solid material (v/v).

17. The method of claim 16, wherein, prior to homogenizing the fermentation composition, the fermentation composition is diluted in water to a final concentration of from about 30% to about 35% solid material (v/v).

18. The method of any one of claims 15-17, wherein the fermentation composition is homogenized in one or more steps.

19. The method of claim 18, wherein the fermentation composition is homogenized in from one to five steps.

20. The method of claim 19, wherein the fermentation composition is homogenized in from one to three steps.

21. The method of claim 20, wherein the fermentation composition is homogenized in two steps.

22. The method of any one of claims 18-21, wherein each step comprises homogenizing the fermentation composition at a pressure of from about 400 bar to about 1,200 bar.

23. The method of claim 22, wherein each step comprises homogenizing the fermentation composition at a pressure of from about 800 bar to about 1,000 bar.

24. The method of claim 23, wherein each step comprises homogenizing the fermentation composition at a pressure of about 900 bar.

25. The method of any one of claims 18-24, wherein each step comprises homogenizing the fermentation composition at a temperature of between about 5° C. and 70° C.

26. The method of any one of claims 18-25, wherein each step comprises homogenizing the fermentation composition at ambient temperature.

27. The method of any one of claims 18-26, wherein each step comprises homogenizing the fermentation composition at a pH between about 3 and about 9.

28. The method of any one of claims 18-26, wherein each step comprises homogenizing the fermentation composition at native pH.

29. The method of any one of claims 11-28, wherein the extracting comprises separating the homogenized fermentation composition resulting from (i) into sediment and supernatant by way of solid-liquid centrifugation.

30. The method of claim 29, wherein, prior to centrifugation of the fermentation composition resulting from (i), the fermentation composition is heated to a temperature of from about 18° C. and about 75° C.

31. The method of claim 29 or 30, wherein, prior to centrifugation of the fermentation composition resulting from (i), the fermentation composition resulting from (i) is diluted in water to a final concentration of from about 20% to about 30% solid material (v/v), optionally wherein the fermentation composition resulting from (i) is diluted in water to a final concentration of about 25% solid material (v/v).

32. The method of any one of claims 29-31, wherein the fermentation composition resulting from (i) is centrifuged at a rate of from about 3,000 revolutions per minute (rpm) to about 5,000 rpm.

33. The method of claim 32, wherein the fermentation composition resulting from (i) is centrifuged at a rate of about 4,100 rpm.

34. The method of any one of claims 29-31, wherein the fermentation composition resulting from (i) is centrifuged with a continuous centrifuge.

35. The method of any one of claims 29-34, wherein the fermentation composition resulting from (i) is centrifuged for from about 5 minutes to about 30 minutes.

36. The method of claim 35, wherein the fermentation composition resulting from (i) is centrifuged for about 15 minutes.

37. The method of any one of claims 11-36, wherein the extracting comprises demulsifying the supernatant obtained from (ii).

38. The method of claim 37, wherein the demulsifying comprises contacting the supernatant obtained from (ii) with a surfactant.

39. The method of claim 38, wherein the surfactant is selected from DOWFAX® 2A1, DOWFAX® 3B2, DOWFAX® 8390, DOWFAX® C6L, DOWFAX® C10L, TRITON® QS-15, TRITON® XN-45S, TERGITOL® L62, or any combination thereof.

40. The method of claim 39, wherein the surfactant is DOWFAX® 2A1.

41. The method of any one of claims 38-40, wherein the surfactant is added to the supernatant obtained from (ii) to a final concentration of from about 0.01% to about 5% (v/v).

42. The method of claim 41, wherein the surfactant is added to the supernatant obtained from (ii) to a final concentration of from about 1% to about 2% (v/v).

43. The method of any one of claims 37-42, wherein the demulsifying is performed at a pH of from about 6 to about 8.

44. The method of any one of claims 37-43, wherein the demulsifying is performed at a temperature of from about 50° C. to about 90° C.

45. The method of claim 44, wherein the demulsifying is performed at a temperature of about 70° C.

46. The method of any one of claims 11-45, wherein the extracting comprises separating the demulsified supernatant resulting from (iii) into an aqueous component and an oil component.

47. The method of claim 46, wherein the demulsified supernatant resulting from (iii) is separated into an aqueous component and an oil component by way of liquid-liquid centrifugation.

48. The method of claim 47, wherein the liquid-liquid centrifugation is performed in one or more steps.

49. The method of claim 48, wherein the liquid-liquid centrifugation is performed in two steps.

50. The method of claim 48, wherein the liquid-liquid centrifugation is performed in three steps.

51. The method of claim 48, wherein, in a first liquid-liquid centrifugation step, the demulsified supernatant resulting from (iii) is centrifuged at a temperature of from about 50° C. to about 90° C.

52. The method of claim 51, wherein, in the first liquid-liquid centrifugation step, the demulsified supernatant resulting from (iii) is centrifuged at a temperature of about 70° C.

53. The method of claim 51 or 52, wherein, in the first liquid-liquid centrifugation step, the demulsified supernatant resulting from (iii) is centrifuged at a rate of from about 3,000 rpm to about 5,000 rpm.

54. The method of claim 53, wherein, in the first liquid-liquid centrifugation step, the demulsified supernatant resulting from (iii) is centrifuged at a rate of about 4,100 rpm.

55. The method of claim 51 or 52, wherein, in the first liquid-liquid centrifugation step, the demulsified supernatant resulting from (iii) is centrifuged with a continuous centrifuge.

56. The method of any one of claims 51-55, wherein, in the first liquid-liquid centrifugation step, the demulsified supernatant resulting from (iii) is centrifuged for from about 5 minutes to about 30 minutes.

57. The method of claim 56, wherein, in the first liquid-liquid centrifugation step, the demulsified supernatant resulting from (iii) is centrifuged for about 15 minutes.

58. The method of any one of claims 51-57, wherein, in a second liquid-liquid centrifugation step, the demulsified supernatant resulting from (iii) is centrifuged at a temperature of from about 25° C. to about 70° C.

59. The method of claim 58, wherein, in the second liquid-liquid centrifugation step, the demulsified supernatant resulting from (iii) is centrifuged at a temperature of between about 40° C. and about 50·° C.

60. The method of claim 58 or 59, wherein, in the second liquid-liquid centrifugation step, the demulsified supernatant resulting from (iii) is centrifuged at a rate of from about 3,000 rpm to about 5,000 rpm.

61. The method of claim 60, wherein, in the second liquid-liquid centrifugation step, the demulsified supernatant resulting from (iii) is centrifuged at a rate of about 4,100 rpm.

62. The method of claim 58 or 59, wherein, in the second liquid-liquid centrifugation step, the demulsified supernatant resulting from (iii) is centrifuged with a continuous centrifuge.

63. The method of any one of claims 58-62, wherein, in the second liquid-liquid centrifugation step, the demulsified supernatant resulting from (iii) is centrifuged for from about 5 minutes to about 30 minutes.

64. The method of claim 63, wherein, in the second liquid-liquid centrifugation step, the demulsified supernatant resulting from (iii) is centrifuged for about 15 minutes.

65. The method of any one of claims 50-64, wherein, in a third liquid-liquid centrifugation step, the demulsified supernatant is centrifuged using a polishing centrifuge.

66. The method of any one of claims 48-65, wherein, between each of the liquid-liquid centrifugation steps, the oil component obtained is mixed with an aqueous solution comprising a base.

67. The method of claim 66, wherein the base is a hydroxide salt.

68. The method of claim 67, wherein the base is selected from NaOH, LiOH, KOH, and Ca(OH)₂.

69. The method of claim 68, wherein the base is NaOH.

70. The method of any one of claims 66-69, wherein the concentration of OH⁻ in the aqueous solution is about 1 M.

71. The method of any one of claims 66-70, wherein the concentration of OH⁻ in the aqueous solution is between about 0.0001 M and about 10 M.

72. The method of any one of claims 47-71, wherein the oil component is mixed with the aqueous solution comprising the base of a ratio between about 0.5:1 to 1:0.1 (oil to aqueous solution, v/v).

73. The method of any one of claims 66-72, wherein the oil component is mixed with the aqueous solution comprising the base at a ratio of about 1:1 (oil to aqueous solution, v/v).

74. The method of any one of claims 66-73, wherein the oil component is mixed with the aqueous solution comprising the base at a temperature of from about 20° C. to about 80° C.

75. The method of claim 74, wherein the oil component is mixed with the aqueous solution comprising the base at a temperature of about 40° C.

76. The method of any one of claims 66-75, wherein the oil component is mixed with the aqueous solution comprising the base for from about 0.1 hours to 10 hours.

77. The method of claim 76, wherein the oil component is mixed with the aqueous solution comprising the base for from about 0.1 hours to about 2 hours.

78. The method of claim 77, wherein the oil component is mixed with the aqueous solution comprising the base for about 1 hour.

79. A method of purifying squalene from an extraction composition, wherein the extraction composition comprises squalene having previously been extracted from a squalene source, the method comprising:
(a) optionally evaporating the squalene from the extraction composition; and
(b) purifying the squalene resulting from (a) by way of chromatography.

80. A method of purifying squalene from an extraction composition, the method comprising:
(a) providing a composition comprising squalene having previously been extracted from a squalene source;
(b) optionally evaporating the squalene from (a); and
(c) purifying the squalene resulting from (b) by way of chromatography.

81. The method of claim 79 or 80, wherein the squalene source is a fermentation source.

82. The method of claim 81, wherein the fermentation source comprises yeast.

83. The method of claim 79 or 80, wherein the squalene source is a plant source.

84. The method of claim 83, wherein the plant source comprises olive, soybean, grape seed, grape, hazelnut, peanut, corn, amaranth, rice, wheat germ, coriander, sesame, or sunflower.

85. The method of claim 79 or 80, wherein the squalene source is an animal source.

86. The method of claim 79 or 80, wherein the squalene source is a fungi source.

87. The method of claim 79 or 80, wherein the fermentation source comprises a stramenopile source.

88. The method of claim 87, wherein the stramenopile source comprises algae.

89. A method of purifying squalene from an extraction composition, wherein the extraction composition comprises squalene having previously been extracted from a fermentation source that has been produced by culturing a population of host cells capable of producing squalene in a culture medium and under conditions suitable for the host cells to produce squalene, the method comprising:
(a) optionally evaporating the squalene from the extraction composition; and
(b) purifying the squalene resulting from (a) by way of chromatography.

90. A method of purifying squalene from a fermentation composition, the method comprising:
(a) providing an extraction composition comprising squalene having previously been extracted from a fermentation source that has been produced by culturing a population of host cells capable of producing squalene in a culture medium and under conditions suitable for the host cells to produce squalene, the method comprising;
(b) optionally evaporating the squalene from (a); and
(c) purifying the squalene resulting from (b) by way of chromatography.

91. The method of any one of claims 1-90, wherein the evaporation step comprises using fractional distillation to isolate squalene.

92. The method of any one of claims 1-90, wherein the evaporation step comprises using simple distillation to isolate squalene.

93. The method of any one of claims 1-92, wherein the evaporation step comprises initially heating the squalene to a temperature of from about 20° C. to about 90° C.

94. The method of claim 93, wherein the evaporation step comprises initially heating the squalene to a temperature of from about 60° C. to about 70° C.

95. The method of any one of claims 1-94, wherein the evaporation step comprises evaporating the squalene at a temperature of from about 150° C. to about 300° C.

96. The method of claim 95, wherein the evaporation step comprises evaporating the squalene at a temperature of from about 200° C. to about 280° C.

97. The method of claim 96, wherein the evaporation step comprises evaporating the squalene at a temperature of from about 200° C. to about 255° C.

98. The method of any one of claims 95-97, wherein the squalene is evaporated under vacuum, optionally wherein the squalene is evaporated at a pressure of between about 0.5 torr and about 5 torr.

99. The method of claim 98, wherein the squalene is evaporated at a pressure of between about 0.7 torr to about 4.0 torr.

100. The method of claim 98, wherein the squalene is evaporated at a pressure of between about 2.0 torr to about 4.0 torr.

101. The method of claim 98, wherein the squalene is evaporated at a pressure of between 0.7 torr to about 2.0 torr.

102. The method of any one of claims 95-101, wherein, following the evaporating, the squalene is condensed and cooled to a temperature of about 70° C. or less.

103. The method of claim 102, wherein, following the evaporating, the squalene is condensed and cooled to a temperature of from about 20° C. to about 70° C.

104. The method of claim 103, wherein, following the evaporating, the squalene is condensed and cooled to a temperature of from about 20° C. to about 25° C.

105. The method of any one of claims 102-104, wherein the squalene is condensed and cooled under vacuum, optionally wherein the squalene is condensed and cooled at a pressure of about 1 torr.

106. The method of any one of claims 102-105, wherein the squalene is condensed and cooled under N₂.

107. The method of any one of claims 1-106, wherein the chromatography comprises exposing the squalene to a polar resin and recovering the squalene from the resin.

108. The method of claim 107, wherein the resin comprises aluminum oxide.

109. The method of claim 108, wherein the aluminum oxide is basic aluminum oxide.

110. The method of claim 108, wherein the aluminum oxide is acidic aluminum oxide.

111. The method of claim 108, wherein the aluminum oxide is neutral aluminum oxide.

112. The method of claim 107, wherein the resin comprises silica.

113. The method of any one of claims 107-112, wherein the resin has an average particle size of from about 50 μm to about 700 μm.

114. The method of claim 113, wherein the resin has an average particle size of from about 50 μm to about 250 μm.

115. The method of claim 113, wherein the resin has an average particle size of from about 300 μm to about 650 μm.

116. The method of any one of claims 1-115 wherein the chromatography is performed using a flow rate of from about 1 bed volumes per hour (BV/hr) to about 5 BV/hr.

117. The method of claim 116, wherein the chromatography is performed using a flow rate of from about 1.5 BV/hr to about 3 BV/hr.

118. The method of claim 117, wherein the chromatography is performed using a flow rate of from about 2 BV/hr to about 2.5 BV/hr.

119. The method of any one of claims 1-118, wherein the chromatography is performed at a temperature of between about 5° C. to about 70° C.

120. The method of any one of claims 1-118, wherein the chromatography is performed at ambient temperature.

121. The method of any one of claims 1-120, wherein an antioxidant is added to the squalene of (c).

122. The method of claim 121, wherein the antioxidant is Vitamin E.

123. The method of claim 122, wherein the Vitamin E is present at a concentration ranging from about 100 to about 1000 ppm.

124. The method of claim 123, wherein the Vitamin E is present at a concentration of about 500 ppm.

125. The method of any one of claims 1-124, wherein the host cell is a yeast cell.

126. The method of claim 125, wherein the yeast cell is S. cerevisiae.

127. The method of any one of claims 1-126, wherein the squalene is isolated from the fermentation composition with a purity of from about 90% (w/w) to about 100% (w/w).

128. The method of claim 127, wherein the squalene is isolated from the fermentation composition with a purity of from about 95% (w/w) to about 100% (w/w).

129. The method of claim 128, wherein the squalene is isolated from the fermentation composition with a purity of from about 99.5% (w/w) to about 100% (w/w).

130. A composition comprising squalene, wherein the composition is produced by the method of any one of claims 1-129.

131. The composition of claim 130, wherein the squalene has a purity of from about 90% (w/w) to about 100% (w/w).

132. The composition of claim 131, wherein the squalene has a purity of from about 95% (w/w) to about 100% (w/w).

133. The composition of claim 132, wherein the squalene has a purity of from about 99.5% (w/w) to about 100% (w/w).

134. A pharmaceutical composition comprising squalene and one or more pharmaceutically acceptable carriers, diluents, or excipients, wherein the purity of the squalene is from about 99.5% (w/w) to about 100% (w/w).

135. The composition of any one of claims 130-134, wherein the squalene is present with one or more impurities, and wherein the one or more impurities are present in a concentration of about 0.5% (w/w) or less.

136. The composition of claim 135, wherein the one or more impurities are present in a concentration of about 0.4% (w/w) or less, optionally wherein the one or more impurities are present in a concentration of about 0.3% (w/w) or less, optionally wherein the one or more impurities are present in a concentration of about 0.2% (w/w) or less, optionally wherein the one or more impurities are present in a concentration of about 0.1% (w/w) or less.

137. The composition of claim 135 or 136, wherein the one or more impurities comprise a fatty acid and/or a sterol.

138. An adjuvant formulation comprising squalene produced by the method of any one of claims 1-129 and a pharmaceutically acceptable carrier, diluent, or excipient.

139. An adjuvant formulation comprising the composition of any one of claims 130-137.

140. A vaccine comprising a therapeutically or prophylactically effective amount of the adjuvant formulation of claim 138 or 139 and an antigen.

141. A vaccine comprising the squalene produced by the method of any one of claims 1-130 and an antigen.

142. The vaccine of claim 140 or 141, wherein the antigen is a protein expressed by a virus.

143. The vaccine of claim 140 or 141, wherein the antigen is encoded by a nucleic acid molecule encoding a protein expressed by a virus.

144. The vaccine of claim 143, wherein the nucleic acid molecule is a deoxyribonucleic acid (DNA) or a ribonucleic acid (RNA) molecule.

145. The vaccine of any one of claims 142-144, wherein the virus is selected from influenza virus, hepatitis A virus, hepatitis B virus, hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabia virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus, rhinovirus, mumps virus, poliovirus, human enterovirus, coxsackievirus, human papilloma virus, adeno-associated virus, astrovirus, JC virus, BK virus, SV40 virus, Norwalk virus, rotavirus, human immunodeficiency virus (HIV), human T-lymphotropic virus, SARS-COV-2, MERS-COV, SARS-COV, OC43, and HKU1.

146. The vaccine of claim 140 or 141, wherein the antigen is a protein expressed by a bacterium.

147. The vaccine of claim 140 or 141, wherein the antigen is encoded by a nucleic acid molecule encoding a protein expressed by a bacterium.

148. The vaccine of claim 147, wherein the nucleic acid molecule is a DNA or RNA molecule.

149. The vaccine of any one of claims 146-148, wherein the bacterium belongs to a genus selected from Mycobacterium, Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus.

150. The vaccine of claim 140 or 141, wherein the antigen is a protein expressed by a parasite.

151. The vaccine of claim 140 or 141, wherein the antigen is encoded by a nucleic acid molecule encoding a protein expressed by a parasite.

152. The vaccine of claim 151, wherein the nucleic acid molecule is a DNA or RNA molecule.

153. The vaccine of any one of claims 150-152, wherein the parasite is selected from Plasmodium malariae, Plasmodium vivax, Plasmodium ovale, Plasmodium falciparum, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Trichomonas vaginalis, and Histomonas meleagridis, Richuris trichiura, Ascaris lumbricoides, Enterobius vermicularis, Ancylostoma duodenale, Necator americanus, Strongyloides stercoralis, Wuchereria bancrofti, Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus.

154. The vaccine of claim 140 or 141, wherein the antigen is a protein expressed by a cancer cell.

155. The vaccine of claim 140 or 141, wherein the antigen is encoded by a nucleic acid molecule encoding a protein expressed by a cancer cell.

156. The vaccine of claim 155, wherein the nucleic acid molecule is a DNA or RNA molecule.

157. The vaccine of any one of claims 154-156, wherein the protein is selected from gp100, Kallikrein 4, PBF, PRAME, WT1, HSDL1, Mesothelin, NY-ESO-1, CEA, p53, Her2/Neu, EpCAM, CA125, Folate receptor α, Sperm protein 17, TADG-12, MUC-1, MUC-16, L1CAM, HERV-K-MEL, KK-LC-1, KM-HN-1, LAGE-1, Sp17, TAG-1, TAG-2, ENAH (hMena), mammaglobin-A, NY-BR-1, BAGE-1, MAGE-A1, MAGE-A2, MAGE-A4, MAGE-A6, MAGE-A9, MAGE-A10, MAGE-A12, MAGE-C2, mucink, SSX-2, SSX-4, TRAG-3, c-myc, cyclin B1, p62, Survivin, CD45, DKK1, RU2AS, Telomerase, K-ras, G250, Hepsin, Intestinal carboxyl esterase, α-foetoprotein, M-CSF, PSMA, CASP-5, COA-1, OGT, OS-9, TGF-βRII, gp70, CALCA, CD274, mdm-2, α-actinin-4, Elongation factor 2, ME1, NFYC, GAGE-1/2/8, GAGE-3/4/5/6/7, XAGE-1b/GAGED2a, STEAP1, PAP, PSA, FGF5, hsp70-2, ARTC1, B-RAF, β-catenin, Cdc27, CDK4, CDK12, CDKN2A, CLPP, CSNK1A1, FN1, GAS7, GPNMB, HAUS3, LDLR-fucosyltransferase, MART2, MATN, MUM-1, MUM-2, MUM-3, neo-PAP, Myosin class I, PPP1R3B, PRDX5, PTPRK, N-ras, RBAF600, SIRT2, SNRPD1, Triosephosphate isomerase, OA1, RAB38/NY-MEL-1, TRP-1/gp75, TRP-2, tyrosinase, Melan-A/MART-1, GnTVf, LY6K, and NA88-A.

158. A method of inducing an antigen-specific immune response in a subject, the method comprising administering to the subject the vaccine of any one of claims 140-157.

159. The method of claim 158, wherein the subject is a mammal.

160. The method of claim 159, wherein the mammal is a human.
